(19) United States
(12) Patent Application Publication
(10) Pub. No.: US 2025/0090573 A1
(43) Pub. Date: Mar. 20, 2025

(54) COMPOSITIONS FOR DELIVERING NITRIC OXIDE AND FLUORIDE AND METHODS FOR MAKING AND USING THE SAME

(71) Applicant: University of Georgia Research Foundation, Inc., Athens, GA (US)

(72) Inventors: Hitesh Handa, Athens, GA (US); Lori Marie Estes Bright, Ball Ground, GA (US); Elizabeth J. Brisbois, Athens, GA (US); Mark Garren, Athens, GA (US)

(21) Appl. No.: 18/729,242

(22) PCT Filed: Jan. 17, 2023
(86) PCT No.: PCT/US2023/060751

§ 371 Date: Jul. 16, 2024

Related U.S. Application Data
(60) Provisional application No. 63/266,878, filed on Jan. 18, 2022.

(51) Int. Cl. A61K 31/765; A61K 9/06; A61K 31/095; A61K 31/734; A61P 31/04

(52) U.S. Cl. CPC A61K 31/765; A61K 9/06; A61K 31/095; A61K 31/734; A61P 31/04

(57) ABSTRACT

Described herein are compositions for delivering fluoride ions and nitric oxide to a subject. The compositions described herein include a poloxamer, an alginate, a nitric oxide releasing compound, fluoride ions, and calcium ions. In one aspect, the compositions are hydrogels. The compositions described herein can eradicate oral pathogens and prevent demineralization of teeth in a subject.

### CROSS-REFERENCE TO RELATED APPLICATIONS

This application claims the benefit of and priority to co-pending U.S. Provisional Patent Application No. 63/266,878, filed on Jan. 18, 2022, the contents of which are incorporated by reference herein in their entireties.

### BACKGROUND

Periodontitis, also known as gum disease, propagates from the infection at the gum-bone tissue interface in oral cavities. The process often leads to dental caries, or tooth decay, which is the leading cause of oral pain and tooth loss.¹ Deterioration is caused by several factors, the most prominent being decay-causing bacteria in the mouth producing strong acids that attack enamel and induce cavity formation. If the cavity is left untreated it can cause pain and infection and can only be treated by filling the decayed area with composite resin, covering the damaged area with a porcelain crown, or via a root canal, where the damaged nerves and roots are removed.² Depending on the advancement and severity of the cavity, it may even be necessary to extract the tooth or teeth, which can be very costly. In fact, dental expenditures in the U.S. reached $124 billion in 2016,³ an unnecessary amount considering dental caries can be prevented and even reversed with proper care and treatment. Such preventative measures include good oral hygiene, limiting food high in sugars and starches, seeing a dentist for regular check-ups, and using fluoride, a mineral that can prevent, stop, and reverse tooth decay.⁴

### SUMMARY

Described herein are compositions for delivering fluoride ions and nitric oxide to a subject. The compositions described herein include a poloxamer, an alginate, a nitric oxide releasing compound, fluoride ions, and calcium ions. In one aspect, the compositions are hydrogels. The compositions described herein can eradicate oral pathogens and prevent demineralization of teeth in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1C show an overview of the NO-releasing hydrogel system. Fabrication (A) of Pluronic-alginate (PA) hydrogels begins with preparation of a Pluronic sol blended with GSNO at different weight ratios. This sol is combined with an alginate sol (with NaF incorporated) slightly below room temperature. The mixture is cast into dishes, heated to 37° C., and crosslinked via rapid application of calcium chloride solution. The as-prepared hydrogel (B) forms a gel nanostructure of organized domains of Pluronic micelles separated by a crosslinked network of alginate with variable distribution in domain size and number of micelles. Amine fluorides are capable of fluoride release and elicit an antimicrobial effect through monolayer adsorption of the surfactant onto enamel with a bacteriocidic effect from excess cationic charge from the lipophilic ammonium salt. The nonionic surfactant nature of Pluronic micelles loaded with GSNO (C) suggests a similar mechanism of action is possible, with GSNO having several primary and secondary amines that can become protonated and balance anionic fluoride ions under physiological conditions. Theoretical pKₐ's calculated using Marvin (ChemAxon) based on atomic partial charge distribution following Brönsted's rule.

FIGS. 2A-2D show the physical and mechanical characterization of PA gels. (A) SEM imaging of freeze-dried gels shows the porous nanostructure of the material. (B) Controlled shear rate testing of PA gels at 25° C. demonstrates the shear-thinning behavior with a shear-rate dependent viscosity function based on stability of the gel nanostructure with an alginate network organizing Pluronic micelle domains. (C) Uniaxial compressive testing of the PA gels demonstrates the tunable mechanical properties based on GSNO and NaF loading. D) Swelling capacity studies further demonstrate the decreased mechanical properties of the hydrogels with the incorporation of fluoride and GSNO. Data presented as mean±standard deviation (N=5 per sample group). Statistical significance shown as * (p<0.05), ** (p<0.01), *** (p<0.001), and **** (p<0.0001).

FIGS. 3A-3D show and overview of NO release from fabricated hydrogels. NO evolution is achieved (A) from the homolytic cleave of the nitrosothiol bond in GSNO in the presence of heat, light, or metal ions under physiological conditions. Catalytically depleting all GSNO in the gels showed (B) the total loading of NO adjusted for the mass of the gel based on the formulation. The fabricated gels exhibited sustained, physiologically active release of NO during (C) the first four hours after fabrication and crosslinking. Further long-term study of the gels demonstrated (D) preserved NO release over fourteen days when fabricated, crosslinked, and immediately stored at 4° C. Data presented as mean±SD (N=5). Statistical significance shown as not significant (ns, p>0.05) and **** (p<0.0001).

FIGS. 4A-4C show the analysis of fluoride in hydrogels. EDS-SEM surface analysis (A) and quantification (B) of freeze-dried hydrogels for nitrogen (corresponding to GSNO) and fluoride (corresponding to NaF). Total time-dependent fluoride release (C) was further measured for PA-F and PA-F-G₃₀ gels incubated in artificial saliva at 37° C. Data represents mean±SD (N=5 per sample type). Statistical significance shown as *** (p<0.001) and **** (p<0.0001).

FIGS. 5A-5C show the analysis of antibacterial efficacy of PA gels against S. mutans. (A) The 4-hour viability of S. mutans was determined following direct contact exposure to gel coupons. Biofilms of S. mutans were further grown on hydroxyapatite discs for 36 h and treated with gels for 24 h, after which (B) extracellular polymeric substance was quantified using crystal violet assay and (C) bacteria adhesion was monitored via SEM. Scale bars correspond to 40 μm in the left column and 10 μm in the right column. Data presented as mean±SD (N=3 per sample type). Statistical significance shown as * (p<0.05) and ** (p<0.01).

FIGS. 6A-6C show demineralization prevention potential of PA gels in a hydroxyapatite disc model. Discs (A) were treated with gels for 1 h followed by rinsing and exposure to an acidic demineralization solution, rinsing, and lastly SEM imaging. Quantification (B) of the porous surface structures shown in (C) demonstrated decreased pore formation on discs treated with fluoride-containing gels, suggesting fluorapatite formation. These studies confirmed decreased percent porosity of the hydroxyapatite discs with prior treatment with fluoride-containing gels. Data presents mean±SD (N=3 per treatment group). Statistical significance expressed as * (p<0.05) and *** (p<0.001).

FIGS. 7A-7C show cytocompatibility evaluation of fabricated gels in direct contact exposure against (A) hFoB 1.19 human osteoblasts and (B) human gingival fibroblasts (HGF) over 4 and 24 h. Corresponding images (C) of cell cultures after 4 h incubation under direct exposure with the hydrogels. Data presented as mean±SD (N=3 per sample type).

FIGS. 8A-8B show ATR-FTIR spectroscopy of as prepared GSNO and freeze-dried gels. (A) Synthesized GSNO shows characteristic NO bond vibration band at 1477 cm⁻¹. (B) Fabricated gels show nearly identical polymeric composition and bond vibrational stretching, with some phase shifting of the v(—COO⁻) band to lower wavenumbers at 1355 cm⁻¹ in crosslinked alginate.

FIG. 9 shows the full EDS-SEM surface analyses of freeze-dried hydrogels from several of the formulations.

FIGS. 10A-10D show the stress-strain curves generated from uniaxial compressive testing of (A) PA, (B) PA-F, (C) PA-G₃₀, and (D) PA-F-G₃₀ gels.

FIG. 11 shows the standard curve for electrochemical quantification of fluoride ions in artificial saliva solution.

FIGS. 12A-12F show the evaluation of GSNO and precursor sol materials in in vitro models of human cell cytocompatibility. Relative viability of (A) hFOB 1.19 human osteoblasts and (B) human gingival fibroblasts (HGF) challenged against synthesized GSNO. Relative cell viability after 4 h of exposure to sol materials in (C) hFoB 1.19 and (D) HGF. Further evaluation of cellular viability after 24 h exposure to sol materials in (E) hFOB 1.19 and (F) HGF.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions and Abbreviations

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent” includes, but are not limited to, mixtures or combinations of two or more such solvents, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “hydrogel” is defined herein as non-fluid colloidal network or polymer network that is expanded throughout its whole volume by a fluid. IUPAC. Compendium of Chemical Terminology, 2nd ed. (the “Gold Book”). Compiled by A. D. McNaught and A. Wilkinson. Blackwell Scientific Publications, Oxford (1997).

The term “oral cavity” is the mouth of the subject and includes the teeth, hard late, soft plate, uvula, tonsils, floor of the mouth, tongue, inferior labial frenulum, retromolar trigone, palatine arch, glossopalatine arch, gingiva, and superior labial frenulum.

The term “periodontal disease” also known as gum disease, is a set of inflammatory conditions affecting the tissues surrounding the teeth. In its early stage, called gingivitis, the gums become swollen, red, and may bleed. It is a cause of tooth loss for adults. In its more serious form, called periodontitis, the gums can pull away from the tooth, bone can be lost, and the teeth may loosen or fall out.

### Compositions

In accordance with the purpose(s) of the present disclosure, described herein are compositions for delivering fluoride ions and nitric oxide to a subject. The compositions described herein include a poloxamer, an alginate, a nitric oxide releasing compound, fluoride ions, and calcium ions.

### Poloxamers

The compositions described herein include one or more poloxamers. Poloxamers are nonionic triblock copolymers composed of a central hydrophobic chain of polyoxypropylene (e.g., (poly (propylene oxide)) flanked by two hydrophilic chains of polyoxyethylene (e.g., poly (ethylene oxide)). In one aspect, poloxamer has the formula

HO(C₂H₄O)_{b}(C₃H₆O)ₐ(C₂H₄O)_{b}OH
wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from about 1 kDa to about 20 kDa, or 1 kDa, 2 kDa, 3 kDa, 4 kDa, 5 kDa, 6 kDa, 7 kDa, 1 kDa, 8 kDa, 9 kDa, 10 kDa, 11 kDa, 12 kDa, 13 kDa, 14 kDa, 15 kDa, 16 kDa, 17 kDa, 18 kDa, 19 kDa, or 20 kDa, where any value can be a lower and upper endpoint of a range (e.g., 5 kDa to 15 kDa). Poloxamers useful herein are sold under the tradename Pluronic® manufactured by BASF. Non-limiting examples of poloxamers useful herein include, but are not limited to, those in the table below.

In one aspect, the poloxamer has a molecular weight from about 12,000 Da to about 13,000 Da, about 175 to about 225 ethylene oxide units, and about 50 to about 75 propylene oxide units. In another aspect, the poloxamer is Pluronic® F127.

| | | Average number | Average number | |
|---|---|---|---|---|
| Copolymer | MW | of EO units | of PO units | CMC (M) |
| F68 | 8,400 | 152.73 | 28.97 | 4.8 × 10⁻⁴ |
| P103 | 4,950 | 33.75 | 59.74 | 6.1 × 10⁻⁶ |
| P105 | 6,500 | 73.86 | 56.03 | 6.2 × 10⁻⁶ |
| P123 | 5,750 | 39.2 | 69.4 | 4.4 × 10⁻⁶ |
| F127 | 12,600 | 200.45 | 65.17 | 2.8 × 10⁻⁶ |
| L121 | 4,400 | 10.00 | 68.28 | 1.1 × 10⁻⁶ |

In one aspect, the amount of poloxamer is from about 10% weight/volume (w/v) to about 30% weight/volume (w/v) of the composition. In another aspect, the amount of poloxamer is 10% weight/volume, 12% weight/volume, 14% weight/volume, 16% weight/volume, 18% weight/volume, 20% weight/volume, 22% weight/volume, 24% weight/volume, 26% weight/volume, 28% weight/volume, or 30% weight/volume, where any value can be a lower and upper endpoint of a range (e.g., 6% weight/volume to 24% weight/volume).

### Alginates

Alginates are the anionic form of alginic acid or algin. Alginic acid is a linear copolymer with homopolymeric blocks of (1→4)-linked β-D-mannuronate (M) and α-L-guluronate (G) residues, respectively, covalently linked together in different sequences or blocks. Alginates useful herein are commercially-available or can be synthesized accordingly. The alginate includes a counterion. In one aspect, the counterion is an alkali metal ion or an alkaline earth metal ion. In one aspect, the alginate is sodium alginate.

In one aspect, the alginate has a viscosity of from about 5 centipoise to about 50 centipoise. In another aspect, the alginate has a viscosity of 5 centipoise, 10 centipoise, 15 centipoise, 20 centipoise, 25 centipoise, 30 centipoise, 35 centipoise, 40 centipoise, 45 centipoise, or 50 centipoise, where any value can be a lower and upper endpoint of a range (e.g., 15 centipoise to 25 centipoise).

In one aspect, the amount of alginate is from about 0.1% weight/volume (w/v) to about 5% weight/volume (w/v) of the composition. In another aspect, the amount of alginate is 0.1% weight/volume, 0.5% weight/volume, 1.0% weight/volume, 1.5% weight/volume, 2.0% weight/volume, 2.5% weight/volume, 3.0% weight/volume, 3.5% weight/volume, 4.0% weight/volume, 4.5% weight/volume, or 5.0% weight/volume, where any value can be a lower and upper endpoint of a range (e.g., 0.1% weight/volume to 0.3% weight/volume).

### Nitric Oxide Releasing Compounds

The nitric oxide releasing compound is a compound that possesses one or more nitric oxide groups, wherein nitric oxide is subsequently released from the compound. In one aspect, the nitric oxide releasing compound is a S-nitrosothiol compound. In another aspect, the nitric oxide compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

In one aspect, the amount of the nitric oxide releasing compound is from about 0.1 mg/ml of the composition to about 30 mg/ml of the composition. In another aspect, the amount of the nitric oxide releasing compound is 0.1 mg/ml, 0.5 mg/ml, 1.0 mg/ml, 5.0 mg/ml, 10.0 mg/ml, 15.0 mg/ml, 20.0 mg/ml, 25.0 mg/ml, or 30.0 mg/ml, where any value can be a lower and upper endpoint of a range (e.g., 5.0 mg/ml to 25.0 mg/ml).

### Fluoride and Calcium Ions

The source of the fluoride and calcium ions can be fluoride salts and calcium salts. In one aspect, the fluoride salts and calcium salts are water soluble salts. Examples of fluoride salts useful herein include sodium fluoride (NaF) or ammonium fluoride (NH₄F). In one aspect, the amount of fluoride ions is from about 0.01% weight/volume (w/v) to about 1% weight/volume (w/v) of the composition. In another aspect, the amount of fluoride ions is 0.01% weight/volume, 0.05% weight/volume, 0.10% weight/volume, 0.20% weight/volume, 0.30% weight/volume, 0.40% weight/volume, 0.50% weight/volume, 0.60% weight/volume, 0.70% weight/volume, 0.80% weight/volume, 0.90% weight/volume, or 1.00% weight/volume, where any value can be a lower and upper endpoint of a range (e.g., 0.1% weight/volume to 0.3% weight/volume). In another aspect, the calcium salt is calcium chloride (CaCl₂)) or calcium fluoride (CaF₂).

### Methods for Making the Compositions

Described herein are methods for making the compositions. In one aspect, the compositions are hydrogels. In one aspect, the hydrogel is produced by the process comprising
(a) mixing an alginate, a nitric oxide releasing compound, and a fluoride salt in water to produce a first composition;
(b) mixing a poloxamer with the first composition to produce a second composition comprising a gel; and
(c) applying a solution comprising a calcium salt to the gel to produce the composition.

In one aspect, the alginate is dissolved in deionized water prior to mixing with the nitric oxide releasing compound and a fluoride salt. After the alginate, a nitric oxide releasing compound, and a fluoride salt are mixed in water to produce a first composition, the poloxamer is added to the first composition to produce a second composition. The second composition is mixed for a sufficient time to ensure that all the components in the second composition are evenly or homogeneously dispersed throughout the composition. The second composition subsequently turns into a gel.

After the formation of the hydrogel, the gel is contacted with a solution comprising a dissolved calcium salt. In one aspect, the calcium solution is one or more calcium salts dissolved in water. In another aspect, the gel is in contact with the solution comprising the calcium salt for about 10 minutes to about 120 minutes at 20° C. to about 30° C. in the absence of light. Non-limiting procedures for making the compositions described herein are provided in the Examples.

The relative amounts of alginate and poloxamer can alter or modify the physical properties of the compositions described herein. In one aspect, the weight/volume (w/v) ratio of poloxamer to alginate is from 1:1 to 20:1, or 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1, where any value can be a lower and upper endpoint of a range (e.g., 5:1 to 14:1).

Not wishing to be bound by theory, the combination of poloxamer and alginate creates a composite gel with increased erosion resistance while demonstrating a ‘stronger,’ more stable alginate gel interspersed within a ‘softer’ thermosensitive poloxamer matrix. Through a dual crosslinking process, the poloxamer can be thermally set within an incubator while alginate forms an interpenetrating network (IPN) within the poloxamer pores and is then crosslinked with the calcium ions. The utilization of crosslinked alginate within the thermosensitive poloxamer matrix allows for more stable nitric oxide and fluoride release without compromising biocompatibility.

As will be discussed further below, the nitric oxide releasing compound is sensitive to light, which will ultimately cause the release of nitric oxide from the composition. In one aspect, the compositions are stored in the absence of light in order enhance shelf life and the efficiency of the compositions with respect to the amount of nitric oxide that can be released from the composition.

In another aspect, the compositions can include a compound that will reduce or prevent the decomposition of the nitric oxide releasing compound. In one aspect, the compound can be a biocompatible metal chelator such as, for example, an organic polyamine (i.e., an organic compound having two or more amine groups). Examples of metal chelators useful herein include, but are not limited to, ethylenediaminetetraacetic acid (EDTA) or bis(3-aminopropyl) amine (dipropylenetriamine (DPTA). In one aspect, the metal chelator is mixed with the alginate, the nitric oxide releasing compound, and the fluoride salt.

### Kits

Described herein are kits that include all of the components needed to produce the compositions described herein. In one aspect, the kit includes
(a) an alginate in dry form;
(b) a dry mixture comprising a poloxamer, a nitric oxide releasing compound, fluoride salt, and a calcium salt;
(c) water; and
(d) instructions for preparing a composition.

The dry form of alginate and poloxamer, a nitric oxide releasing compound, fluoride salt, and a calcium salt can be a powder (e.g., a lyophilized powder). The powders can be stored indefinitely in the absence of light until they are ready for use. In one aspect, when it is time to use the composition, water can be added separately to the dry alginate and the dry mixture of the poloxamer, nitric oxide releasing compound, fluoride salt, and calcium salt to produce two separate compositions. The compositions are then added together and mixed for a sufficient time to produce the compositions (e.g., the hydrogel). In certain aspects, the kits can include applicators for administering the composition to the subject. In one aspect, when the composition is a hydrogel, the kit includes a syringe or a mouthpiece for delivering the hydrogel to the oral cavity of the subject.

### Methods of Use

The demineralization and breakdown of tooth enamel is characterized by a condition called dental caries, or tooth decay, that is caused by two main factors: (1) highly acidic food intake without proper oral hygiene, and (2) overactive oral bacteria generating acidic metabolic byproducts. Fluoride treatments have been shown to help rebuild the hydroxyapatite structures that make up 98% of enamel, but do not tackle the bacterial overload that continues to threaten future demineralization.

The root of dental caries lies in the overactivity of bacteria on gums and teeth. Streptococcus mutans (S. mutans) and other dental pathogens colonize on the surface of teeth and form biofilms composed of protein, DNA, and polysaccharides. These biofilms, known as dental plaque, act as a protective barrier against antimicrobial treatments and allow the bacteria to proliferate uncontrolled. Although proper dental hygiene and regular brushing can keep these bacteria in balance and at bay, neglect of oral care can lead to excess plaque and in turn, overactive bacteria.

The compositions described herein address these issues, where the compositions are effective in delivering fluoride ions and nitric oxide to the oral cavity of a subject. The compositions possess dual functionality by concurrently releasing fluoride ions that help rebuild the enamel of teeth as well as nitric oxide that can kill bacteria in the oral cavity. Thus, the compositions are effective in treating or preventing bacterial infections, reducing or prevention the formation of biofilms in the oral cavity, and treating or preventing a periodontal disease in a subject.

The release pattern of the fluoride ions and nitric oxide from the composition can be modified or tuned depending upon, among other things, the relative amounts of poloxamer and alginate used to prepare the compositions. The duration of the release of the fluoride ions and nitric oxide can be in the range from about 0.5 minutes to 24 hours. In the case of nitric oxide release, the release pattern can be further modified by exposing the composition to visible light. In one aspect, the composition can be exposed to visible light in the range of from about 350 nm to about 550 nm, or about 350 nm, 375 nm, 400 nm, 425 nm, 450 nm, 475 nm, 500 nm, 425 nm, or 550 nm at an intensity of from 10% to 100%, where any value can be a lower and upper endpoint of a range (e.g., 450 nm to 475 nm). When exposed to visible light, the compositions provide close to instantaneous release of nitric oxide from the composition. The composition can be exposed to visible prior to and/or after administration of the composition to the oral cavity of the subject.

The compositions described herein can be administered to the cavity of the subject using a number of techniques. In one aspect, the composition can be formulated as a hydrogel then applied to the teeth and gums of the subject by a syringe. In another aspect, the hydrogel can be applied to a mouthpiece and subsequently inserted into the oral cavity. In this aspect, all the teeth and gums are exposed to the hydrogel. In certain aspects, the mouthpiece can be made of a transparent or semi-transparent material such that mouthpiece can be exposed to visible light to enhance the release of nitric oxide from the composition. The compositions can be used in the home of the subject or a dentist's office.

### Aspects

Aspect 1. A composition comprising a poloxamer, an alginate, a nitric oxide releasing compound, fluoride ions, and calcium ions.
Aspect 2. The composition of Aspect 1, wherein the poloxamer has the formula

HO(C₂H₄O)_{b}(C₃H₆O)ₐ(C₂H₄O)_{b}OH
wherein a is from 10 to 100 and b is from 5 to 250, 10 to 225, 20 to 200, 50 to 200, 100 to 200, or 150 to 200.
Aspect 3. The composition of Aspect 1 or 2, wherein the poloxamer has a molecular weight of from about 1 kDa to about 20 kDa.
Aspect 4. The composition in any one of Aspects 1-3, wherein the poloxamer has a molecular weight from about 12,000 Da to about 13,000 Da, about 175 to about 225 ethylene oxide units, and about 50 to about 75 propylene oxide units.
Aspect 5. The composition in any one of Aspects 1-4, wherein the poloxamer is from about 10% weight/volume (w/v) to about 30% weight/volume (w/v) of the composition.
Aspect 6. The composition in any one of Aspects 1-5, wherein the alginate comprises an alkali metal alginate or an alkaline earth metal alginate.
Aspect 7. The composition in any one of Aspects 1-5, wherein the alginate comprises sodium alginate.
Aspect 8. The composition in any one of Aspects 1-7, wherein the alginate has a viscosity of from about 5 centipoise to about 50 centipoise.
Aspect 9. The composition in any one of Aspects 1-8, wherein the alginate is from about 0.1% weight/volume (w/v) to about 5% weight/volume (w/v) of the composition.
Aspect 10. The composition in any one of Aspects 1-9, wherein the weight/volume (w/v) ratio of poloxamer to alginate is from 1:1 to 20:1.
Aspect 11. The composition in any one of Aspects 1-10, wherein the nitric oxide releasing compound is a S-nitrosothiol compound.
Aspect 12. The composition in any one of Aspects 1-10, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.
Aspect 13. The composition in any one of Aspects 1-10, wherein the nitric oxide releasing compound is S-nitrosoglutathione.
Aspect 14. The composition in any one of Aspects 1-13, wherein the nitric oxide releasing compound is from about 0.1 mg/ml of the composition to about 30 mg/ml of the composition.
Aspect 15. The composition in any one of Aspects 1-14, wherein the fluoride ions are from about 0.01% weight/volume (w/v) to about 1% weight/volume (w/v) of the composition.
Aspect 16. The composition in any one of Aspects 1-14, wherein the fluoride ions are derived from sodium fluoride.
Aspect 17. A composition produced by the process comprising
   (a) mixing an alginate, a nitric oxide releasing compound, and a fluoride salt in water to produce a first composition;
   (b) mixing a poloxamer with the first composition to produce a second composition comprising a gel; and
   (c) applying a solution comprising a calcium salt to the gel to produce the composition.
Aspect 18. The composition of Aspect 17, wherein the fluoride salt comprises sodium fluoride or ammonium fluoride.
Aspect 19. The composition of Aspect 17 or 18, wherein the second composition is heated from about 30° C. to about 40° C. for a duration of from about 5 minutes to about 120 minutes.
Aspect 20. The composition in any one of Aspects 17-19, wherein the calcium salt comprises calcium chloride or calcium fluoride.
Aspect 21. The composition in any one of Aspects 17-20, wherein in step (c) the gel is in contact with the solution comprising the calcium salt for about 10 minutes to about 120 minutes at 20° C. to about 30° C. in the absence of light.
Aspect 22. The composition in any one of Aspects 1-21, wherein the composition further comprises a metal chelator.
Aspect 23. The composition of Aspect 22, wherein the metal chelator comprises ethylenediaminetetraacetic acid (EDTA) or bis(3-aminopropyl) amine (dipropylenetriamine (DPTA).
Aspect 24. The composition in any one of Aspects 1-23, wherein the composition comprises a hydrogel.
Aspect 25. A method for delivering fluoride ions and nitric oxide to the oral cavity of a subject, the method comprising delivering the composition in any one of Aspects 1-24 to the oral cavity of the subject.
Aspect 26. The method of Aspect 25, wherein the composition prevents demineralization of one or more teeth of the subject.
Aspect 27. The method of Aspect 25 or 26, wherein the composition strengthens damaged enamel of one or more teeth of the subject.
Aspect 28. A method for treating or preventing a bacterial infection in an oral cavity of a subject in need thereof comprising administering to the subject the composition in any one of Aspects 1-24.
Aspect 29. A method for treating or preventing a periodontal disease in a subject in need thereof comprising administering to the subject the composition in any one of Aspects 1-24.
Aspect 30. A method for preventing or reducing the formation of biofilm or dental plaque in a subject in need thereof comprising administering to the subject the composition in any one of Aspects 1-24.
Aspect 31. The method in any one of claims 25-30, wherein the composition is administered topically to one or more teeth of the subject, to the gingiva of the subject, or a combination thereof.
Aspect 32. The method in any one of claims 25-31, wherein after the composition is administered to the subject, the composition is exposed to visible light.
Aspect 33. A kit comprising
   (a) an alginate in dry form;
   (b) a dry mixture comprising a poloxamer, a nitric oxide releasing compound, fluoride salt, and a calcium salt;
   (c) water; and
   (d) instructions for preparing a composition.
Aspect 34. The kit of Aspect 33, wherein the kit further comprises an applicator.
Aspect 35. The kit of Aspect 34, wherein the applicator is a syringe or mouthpiece.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

Acetone, calcium chloride dihydrate, ethylenediaminetetraacetic acid (EDTA), magnesium chloride, methanol, Pluronic® F-127, potassium phosphate dibasic, sodium alginate, sodium ascorbate, sodium carboxymethylcellulose, sodium chloride, and sodium nitrite were purchased from Sigma-Aldrich (St. Louis, MO USA). Reduced L-glutathione (GSH) and G-418 sulfate were purchased from Gold Biotechnology (Jersey City, NJ USA). Sodium fluoride was purchased from Himedia Laboratories (West Chester, PA USA). Citric acid was purchased from J.T. Baker (Phillipsburg, NJ USA). Hydrochloric acid (37%) and fetal bovine serum (FBS) were purchased from VWR (Radnor, PA USA). Hydroxyapatite disc coupons were obtained from BioSurface Technologies Corporation (Bozeman, MT USA). All buffers and other aqueous solutions were prepared using 18.2 MQ ultra-pure water using an in-house distillation apparatus from Mettler Toledo (Columbus, OH USA). Phosphate-buffered saline (PBS) containing 2.7 mM KCl, 138 mM NaCl, 1.8 mM KH₂PO₄, and 10 mM Na₂HPO₄ at pH 7.4 was used in all in vitro experiments. Brain heart infusion agar and broth were purchased from Mckesson Medical Surgical (Irving, TX 75039) and Streptococcus mutans (ATCC® 25175™) was purchased from American Type Culture Collection (ATCC, Manassas, VA, USA). Human derived osteoblast cell line hFOB 1.19 (ATCCR CRL-11372™), Primary Gingival Fibroblast, Normal, Human, Adult (HGF) (ATCCR PCS-201-018™), fibroblast basal medium, and corresponding fibroblast growth kit with low serum were also purchased from ATCC. Dulbecco's modified Eagle's medium with Nutrient Mixture F-12 (1:1 by volume) was purchased from Thermo Fisher Scientific (Waltham, MA USA). Trypsin-EDTA was obtained from Corning (Corning, NY USA). The Cell Counting Kit-8 (CCK-8) was procured from Enzo Life Sciences (Farmingdale, NY USA).

### Synthesis of S-Nitrosoglutathione (GSNO, Nitric Oxide Donor)

GSNO was readily synthesized by the acid-catalyzed nitrosation of GSH with sodium nitrite following previous literature.⁴² In brief, GSH is dissolved in 0.5 M HCl solution to a final concentration of 170 mg/ml and chilled in an ice bath for 10 minutes with stirring. An equimolar amount of sodium nitrite is then added, turning the solution a dark red color. The pot is protected from light and allowed to stir for 45 minutes. Afterwards, an excess of chilled acetone is added to precipitate GSNO. The light pink precipitate is further washed with chilled deionized water and acetone to remove unreacted nitrite, filtered, and lyophilized overnight. Lyophilized GSNO was milled to a fine particulate using an IKA Benchtop A 10 Basic Mill (Cole-Parmer; Vernon Hills, IL). GSNO was always protected from light and stored at −20° C. between experiments. Only GSNO batches of greater than 95% purity were used for fabrication of hydrogels, as determined by measurement of NO moles released per mole of GSNO via chemiluminescent-based nitric oxide release analysis.

### Artificial Saliva and Demineralization Solutions

Artificial saliva solution consisted of 1.2 g L⁻¹ KCl, 1 g L⁻¹ sodium carboxymethylcellulose, 0.8 g L⁻¹ NaCl, 0.3 g L⁻¹ K₂HPO₄, 0.1 g L⁻¹ CaCl₂)·2H₂O, and 0.1 g L⁻¹ MgCl₂·6H₂O in deionized water.⁴³⁻⁴⁴ Artificial saliva was thoroughly dissolved and sterilized by steam autoclaving for 45 minutes at 121° C. prior to all experiments. Demineralization solution was comprised of 0.1 M citric acid adjusted to pH=3.45

Fabrication of Hydrogel Networks Four kinds of alginate-Pluronic® F127 hydrogels were prepared: the first contained only alginate (2% w/v) and Pluronic® F127 (18% w/v) (PA), the second one added sodium fluoride (0.2% w/v) (PA-F), the third one added GSNO (PA-Gx), and the fourth added both sodium fluoride (0.2% w/v) and GSNO (PA-F-Gx). GSNO-containing gels were prepared with a sol concentration of 10 (G₁₀), 20 (G₂₀), and 30 mg/ml (G₃₀) (Scheme 1A). The ratio of alginate to Pluronic® F127 was adopted from previous studies.⁴⁰⁻⁴¹ Precursor solutions were first prepared by dissolving sodium alginate in deionized water at 60° C. for 45 minutes. Afterwards, the solution was cooled to room temperature and GSNO and NaF were added at the previously mentioned concentrations. Pluronic® F127 flakes were then added to the samples, which were stored at 4° C. for up to 24 h before use. Prior to casting, solutions were stirred at room temperature for 1 h to ensure complete dissolution of components, after which a proportionate volume of the precursor solution was aliquoted into a 60×15 mm petri dish. Dishes were covered and wrapped in parafilm to retain moisture and incubated at 37° C. for 30 min (thermal gelation of Pluronic F127 starts at 20° C. and concludes near 24° C.).⁴⁶ Afterwards, a CaCl₂) crosslinking solution (1 g L⁻¹ in deionized water) was quickly sprayed onto the gel in aliquots of exactly 100 μL each. The volume of crosslinking solution was approximately equal to the volume of the gel. The gel was left in contact with the crosslinking solution for 2 h at room temperature and protected from light. Afterwards, the remaining solution was aspirated off the gel, which was then cut into individual gels (8 mm in diameter), briefly rinsed 3× with deionized water to remove excess components not swollen into the gel, and gently padded with a nonwoven wipe to remove excess surface moisture. All in vitro bacteria and cell culture experiments followed the same procedure for gel preparation, with the exceptions being that the precursor solution was first UV sterilized for 30 min and the CaCl₂) crosslinking solution was sterile filtered (<0.22 μm).

### Attenuated Total Reflectance-Fourier Transform I (ATR-FTIR)

Spectroscopy ATR-FTIR spectroscopic measurements were performed using a Spectrum Two spectrometer from Perkin Elmer (Greenville, SC USA) in order to determine the chemical functionality of freeze-dried hydrogel samples and precursor materials. Infrared spectra were recorded from 4000-650 cm⁻¹ with a total of 16 scans using a resolution of 4 cm⁻¹. A KBr loading method was used for each analysis. In a representative test, a 1 wt % mixture of analyte was dissolved in anhydrous potassium bromide, casted into a 7-mm die cast, and processed for 5 min at 1.5 tons of applied force. Three independently prepared specimens were analyzed for each sample type. Final spectra were baseline corrected.

### Scanning Electron Microscopy and Energy-Dispersive X-ray Spectroscopy

Microscopy techniques were used to examine surface morphology and composition of each of the four gel types (PA, PA-F, PA-G₃₀, PA-F-G₃₀). After making 8 mm circular punches and rinsing, the samples were lyophilized for ˜6 hours and stored at room temperature shielded from light. Samples were then coated with 10 nm gold-palladium using a Leica sputter coater (Leica Microsystems). Scanning electron microscopy (SEM, FEI Teneo, FEI Co.) was utilized to acquire images of cross-sectional morphology and porosity of the varying gels. An energy-dispersive x-ray spectroscopy system (EDS, Oxford Instruments) was used in conjunction with the SEM setup to perform elemental analysis of the four sample types. Fluorine measurements corresponded to the presence of sodium fluoride. An accelerating voltage of 5.00 kV was used for SEM and 20.00 kV for EDS.

### Rheology Testing

Controlled shear rate tests of the hydrogels were performed using a cone plate rheometer (Brookfield Engineering Laboratories, Middleboro, MA, USA) equipped with a cone-shaped spindle with a cone angle of 0.8° and radius of 2.4 cm. Speed ramping was performed following ISO 3219 standards following a geometric series for shear rate from 0.1 to 100 s⁻¹ with a multiplier of 2.5 and variable hold times to account for transient effects in the low shear regions of the studies. Approximately 500 μL of each crosslinked gel was molded into a cylindrical sample and placed into the apparatus. Studies were performed with a temperature-controlled bottom plate heated to 37° C. A total of five independently prepared samples for each hydrogel formulation were tested.

### Compression Testing

Uniaxial compression testing of the formulated hydrogels was performed with a Mark-10 Series 5 force gauge equipped onto a motorized stand (Mark-10, Copiague, NY USA). For compressive testing, cylindrical samples of the crosslinked hydrogels (approximately 12 mm in diameter and 2 mm in height) were fabricated and tested. Samples were placed between two parallel plates and tested at 25% strain at a rate of 0.166 mm s⁻¹. A total of five independently prepared samples for each hydrogel formulation were tested.

### Swelling Capacity

The swelling capacity of all four gel types was characterized to determine the change in water uptake characteristics with the addition of NaF and GSNO. Hydrogels were fabricated as previously described and 8 mm diameter punches were lyophilized for 6 h. Following lyophilization, gels were weighed (W_{d}) and then soaked in artificial saliva for 1 h or 4 h at 37° C. in the dark. At that time, gels were removed from the artificial saliva, placed on a nonwoven wipe for 5 seconds, flipped, and then weighed (Wₛ). The swelling capacity was calculated using Equation 1.

$$Swelling Capacity ( \% ) = \frac{{w}_{s} - {w}_{d}}{{w}_{d}} * 100 \qquad ( 1 )$$

### GSNO Loading

The relative number of moles of GSNO loaded per mass of hydrogel in PA-Gₓ and PA-F-Gₓ gels were determined via a modified NO loading quantification method using a Sievers chemiluminescence nitric oxide analyzer (NOA) 280i (Boulder, CO 80301).⁴⁷ In the experimental setup, NO gas liberated from solution phase inside an amber glass sample vial is swept by a nitrogen carrier stream into the reaction chamber of the NOA, wherein NO is reacted with ozone from a separate inlet stream and converted into NO₂ in an excited state. Relaxation of this excited state results in emission of photons which are internally detected via a photomultiplier tube. This photon flux is then correlated against a calibration constant (mol NO/PPB×s) established from a 45 PPM NO gas standard to determine the instantaneous NO release with respect to the mass of gel tested (mol NO/mg gel×s).

In a representative study, circular punchouts of the hydrogel films were weighed (˜50 mg each) and placed in an amber glass sample vial supplemented with 3 mL of PBS (1×) without EDTA. Alternating 200 μL injections of 100 mM solutions of copper (II) chloride and sodium ascorbate were added to the sample chamber to stimulate the degradation of the S-nitrosothiol bond on GSNO by Cu¹⁺ ions.⁴⁸ Injections were added until the NO payload was depleted from each sample. A plot of the NO release (mol NO/s) against time(s) was then adjusted for a baseline reading without the sample and integrated over the duration of the experiment to obtain the loading ratio (mmol NO/mg gel).

### NO Release Under Physiological Conditions

The instantaneous release profiles of NO from GSNO loaded hydrogels was determined across several sol concentrations of GSNO with and without NaF incorporation using chemiluminescence-based detection. In a representative study, an 8 mm circular punchout of the hydrogel film is weighed (˜50 mg each) and wrapped in a nonwoven wipe moistened with artificial saliva solution. The wrapped hydrogel is then suspended above PBS (1×) without submerging in an amber glass sample vial placed in a water bath at 37° C. No metal ion catalyst or reducing agent were added. The instantaneous NO release is measured over a 4 h study and corrected against a baseline reading of the instrument. Each gel type was run in triplicate.

### Storage Stability Analysis (28 d)

To assess the storage stability of the NO-releasing GSNO component of the dental gels, PA-G₃₀ and PA-F-G₃₀ gels were fabricated and stored at 4° C. for up to 28 d. Triplicates of gels were removed from storage conditions and tested in the NOA after 0, 1, 7, 14, 21, and 28 d of storage. Gels were discarded after measurement. The NOA setup was identical to that previously described in the prior NO release section. Therefore, this study assessed NO release in simulated physiological conditions at each time point to determine how much NO release capability was lost over time, or how long the gels could be stored at 4° C. and still maintain potency. NO release measurements were recorded as cumulative 1 h release sums (mmol NO/mg gel).

### Fluoride Release

Cumulative fluoride ion release from the hydrogel samples was determined using a fluoride ion selective TruLine electrode from Xylem Incorporated (Rye Brook, NY USA) against a standard calibration curve in artificial saliva developed against a total ionic strength adjustment buffer from YSI Incorporated (Yellow Springs, OH USA). In brief, hydrogel punchouts of known mass (50 mg each) were incubated in 3 mL of artificial saliva solution for corresponding time points of 10 and 60 min at room temperature. Afterwards, the solution was aspirated off and stored at 4° C. until processing. The electric potential was then measured for each sample time point for PA-F and PA-F-G₃₀ gels (n=5 per treatment time, per hydrogel type). A standard calibration curve was developed using sodium fluoride solution by linearly fitting a plot of the average electric potentials to the log 10 of the known fluoride ion concentration in the analytes. From this, the number of moles of fluoride ions released per mass of hydrogel was calculated using Equation 2.

$$\frac{Mole {F}^{-}}{Mass Gel} = {10}^{\frac{Electric Potential - Intercept}{Slope}} \times \frac{Volume of Analyte}{Mass of Gel} \qquad ( 2 )$$

### Bacteria Culture

Viable bacterial colonies were prepared for antimicrobial tests using the following procedure. A single S. mutans colony was isolated, inoculated in BHI broth, and grown to mid log phase at 37° C. and 150 rpm in a shaker incubator. The bacteria suspension was then rinsed with and resuspended in PBS, and then diluted to ˜10⁸ CFU/mL. The diluted suspension of known bacteria counts was then used to study a 4 h bacterial exposure and 24 h treatment of a biofilm (grown for 36 h prior to treatment) with the antibacterial dental gel.

### Planktonic Bacterial Viability Study

A 4 h bacterial viability study was utilized to monitor the antibacterial efficacy of the gels against S. mutans, one of the most common pathogens known to cause dental caries. Sterilized gels of each type (n=3) were placed in a 24 well plate and incubated for 4 h at 37° C. and 150 rpm in a shaker incubator with 1 mL of the bacterial suspension. Following incubation, 100 μL from each well was removed and serial dilutions were performed. Diluted suspensions were plated on BHI agar and placed in an incubator for 48 h. After 48 h of growth, bacterial colonies were counted to determine the number of viable bacteria per mg of hydrogel treatment. Viable CFUs for each sample were calculated using Equation 3, and percentage of bacteria reduction from each treatment versus control was calculated using Equation 4.

$$Viable CFUs per sample = \frac{\begin{matrix}number CFUs per sample \times dilution factor \times \\ vol suspension treated\end{matrix}}{vol suspension plated} \qquad ( 3 )$$

$$\% Reduction in Bacterial Viability = \frac{control CFU {mL}^{- 1} - treatment CFU {mL}^{- 1}}{control CFU {mL}^{- 1}} \times 100 \% \qquad ( 4 )$$

S. mutans Biofilm Dispersal

Crystal violet (CV) staining was utilized to quantify the ability of the NO releasing PA-F-G₃₀ gels to disperse a biofilm grown on a HA disc. Before treatment, HA discs were sonicated in DI water for 30 min to remove any loose particles and then sterilized under UV light for 15 min on each side. Discs were then placed in a 24-well plate and a previously prepared inoculum of S. mutans in BHI media was added to the wells. The plate was sealed and placed in a shaking incubator at 37° C. for 36 h, with media changed every 8-12 h. Following 36 h of biofilm growth, HA discs were removed from the plate, lightly rinsed with 1 mL of PBS and placed in a new well plate. Sterile gels (PA or PA-F-G₃₀, n=4) were then placed on top of the HA discs and 1 mL of PBS was added to the wells. Control discs without gel treatment were also submerged in 1 mL PBS to act as the untreated control. After 24 h of incubation at 37° C. and under shaking conditions the gels were rinsed off the HA discs and the discs were rinsed twice with PBS. One sample from each treatment or control was prepared for SEM imaging, while the remaining three underwent the staining process. Treated and control HA discs were placed in a 48-well plate and 300 μL of 0.1% CV solution was added to each well. After incubation of the plate at room temperature for 15 min each disc was rinsed 4 times with DI water and placed in a new well plate to dry overnight. The next day, 300 μL of 30% acetic acid was added to each well to dissolve the CV for 15 min. Following dissolution, 125 μL from each well was added to a 96-well plate and the absorbance at 540 nm was recorded and used for analysis, with 30% acetic acid used as a blank.

### Demineralization of Hydroxyapatite Enamel Model

The potential of the gels to prevent the demineralization of HA discs was investigated. Before beginning the study, all HA discs were sonicated in DI water for 30 min and rinsed lightly to remove any loose HA particles. Discs were then placed in individual wells of a 24 well plate, covered with an 8 mm in diameter gel disc of the corresponding treatment group (n=3), and 1 mL artificial saliva solution was added to each well. Treatment groups included control (no gel), PA, PA-F, PA-G₃₀, and PA-F-G₃₀. Following HA disc treatment in a shaker incubator (37° C., 150 rpm) for 1 h, gels were removed, and discs were rinsed 3× with DI water. Treated HA discs were then exposed to 1 mL demineralization solution for 30 min in a shaker incubator (37° C., 150 rpm). Demineralization solution was aspirated off and discs were rinsed 3× with DI water and dried overnight in a desiccator. The demineralization of the treated discs, characterized as induced porosity, was compared to untreated HA discs that were not exposed to demineralization solution using ImageJ analysis. The pixel area of pores was compared to the pixel area of the entire HA disc within the image and a percent porosity was calculated. Single blinded review of the images was carried out by three researchers, with final average percent porosity measurements reported from the independent analyses of images from each sample type.

### Mammalian Cell Culture

The cell lines HGF and hFOB 1.19 were cultured for cytocompatibility assessments. HGF cells were cultured in fibroblast basal medium supplemented with the manufacturer's recommended growth kit (2% fetal bovine serum, 50 μg/mL ascorbic acid, 5 μg/mL rh insulin, 1 g/mL hydrocortisone hemisuccinate, 5 ng/mL rh FGF b, and 7.5 mM L-glutamine) and penicillin-streptomycin (10 units/mL and 10 μg/mL, respectively). hFOB 1.19 cells were maintained in a 1:1 mixture of Ham's F12 Medium and Dulbecco's Modified Eagle's Medium supplemented with L-glutamine (2.5 mM), fetal bovine serum (10%) and G418 (0.3 mg/mL). Both cell types were incubated at 37° C. in a 5% CO₂ humified atmosphere. Medium was replaced every 48 h and both cell lines were subcultured once monolayers were 80% confluent. Cells were detached from the flask surface via enzymatic treatment with 0.05% trypsin and 5 mM EDTA for 5 min, with isolation of cell pellets via centrifugation at 200 RCF for 5 min.

### Cellular Cytotoxicity of GSNO and Precursor Sol Materials

The cellular cytotoxicity of GSNO against HGF and hFOB 1.19 cells was tested over 24 h direct contact experiments. In brief, suspensions of the cultured cells (50,000 cells/mL) were seeded (100 μL/well) onto 96-well TC-treated plates. The plates were pre-incubated for 24 h to permit the cells to reach >80% confluency. Afterwards, 10 μL of a GSNO stock solution (GSNO in PBS 1×) or non-crosslinked hydrogel sol was added (n=5) to corresponding wells and the plate was incubated for an additional 24 h. The media in each well was aspirated off and replaced with fresh media to avoid interference from residual GSH and related species. CCK-8 solution (10 μL/well) was then added to each well and the plate was incubated for 2 h. A separate set of wells containing only media and the dye (n=5) were also prepared to account for background readings. The absorbance of each well was measured at 450 nm, adjusted against the average absorbance reading of the wells with only media. Equation 5 was used to calculate the percentage cell viability of a treatment dosage relative to the untreated control as follows:

$$\% Cell Viability = \frac{Adjusted Average {ABS}_{450} of Treated Set}{Adjusted Average {ABS}_{450} of Untreated Set} \times 100 \% \qquad ( 5 )$$

### Cellular Proliferation in the Presence of Hydrogels

The proliferation of HGF and hFoB 1.19 cells against crosslinked gels was also tested via 24 h direct contact experiments to further evaluate the biocompatibility of the gel formulations. In short, suspensions of the cultured cells (50,000 cells/mL) were inoculated (400 μL/well) into 24-well TC-treated plates. After 24 h of incubation, hydrogel film punches (50 mg each) were UV sterilized for 30 min then inserted into corresponding wells (n=5 per type). After an additional 24 h of incubation, the hydrogel and media were aspirated off and replaced with 400 μL of fresh media. CCK-8 dye was added (40 μL/well) to determine the relative proliferation of cells in treated versus untreated samples, with measurements adjusted against blank wells and final cellular viability calculated using Equation 5.

### Statistical Analysis

All data is reported as mean±standard deviation (SD) unless otherwise stated. All statistical analysis was performed using Prism 9.1 (GraphPad Software, San Diego, CA USA). Statistical comparison with respect to control groups was performed using ordinary one-away analysis of variance with corrections for multiple comparisons tests between means of sample groups. Values of p<0.05 were deemed significant.

### Results and Discussion

### Fabrication of Hydrogel Networks

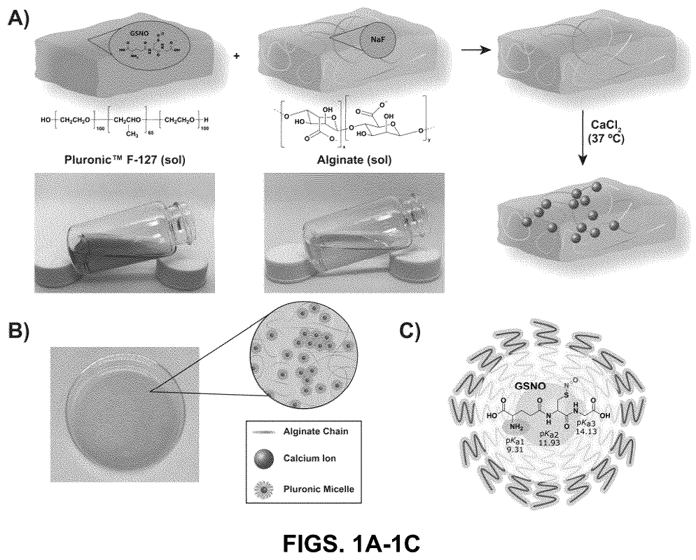

Thermoresponsive hydrogels featuring Pluronic F127 as the major network with an interpenetrating crosslinked alginate backbone have previously shown to be highly biocompatible and display promise for drug release applications.⁴¹ Pluronic F127 is a synthetic poly (oxyethylene)-poly (oxypropylene) block copolymer that is nonionic with thermosensitive properties for micelle formation and stability in aqueous conditions. Combination systems of Pluronic F127 with non-crosslinked alginate have been shown to act as efficacious scaffolds for dental-derived cell encapsulation as well as the enhancement of cell adhesion and promotion of angiogenesis.⁴⁹ In this study, hydrogels of Pluronic F127 with crosslinked alginate (PA) were functionalized with different weight percentages of S-nitrosoglutathione (GSNO) and sodium fluoride (NaF), resulting in the PA-F-Gₓ hydrogels, to support antimicrobial and enamel strengthening properties (FIG. 1). By crosslinking alginate through rapid spraying of a calcium chloride solution, the gelation of the polymer blend was readily optimized with desirable material properties for dental soft tissue applications.

### Materials Characterization

### Attenuated Total Reflectance-Fourier Transform Infrared (ATR-FTIR) Spectroscopy

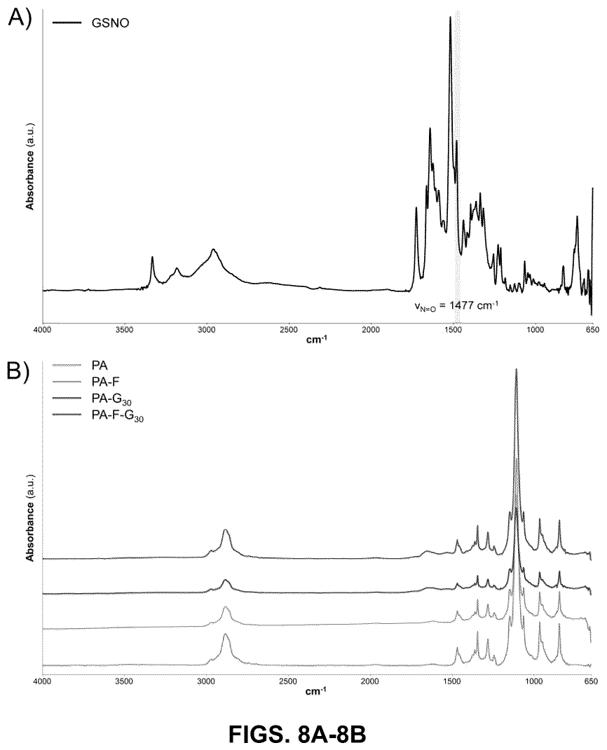

The chemical compositions of synthesized GSNO and lyophilized hydrogels were determined using ATR-FTIR (FIG. 8). Nitrosation of glutathione was confirmed by the emergence of a v(N=O) at 1477 cm⁻¹, with GSNO purity further quantified by NO loading tests for a criterion of greater than 0.95 moles of NO per mol of GSNO tested. Hydrogel network formation with Pluronic F127 and crosslinked alginate were further evidenced by the presence of vibration bands in each gel formulation corresponding to the individual polymer components. Calcium ion crosslinking of alginate resulted in a modest shift in the v(—COO⁻) band from 1422 cm⁻¹ in non-crosslinked alginate to lower wavenumbers at 1355 cm⁻¹, suggesting ionic interaction in agreement with previous reports.⁵⁰ Further characterization confirmed the composition of GSNO and Pluronic F127 in the freeze-dried gel matrices with respect to reference spectra. These results warranted further physical and mechanical testing to investigate the structure and functionality of the hydrogel materials.

### Scanning Electron Microscopy (SEM)

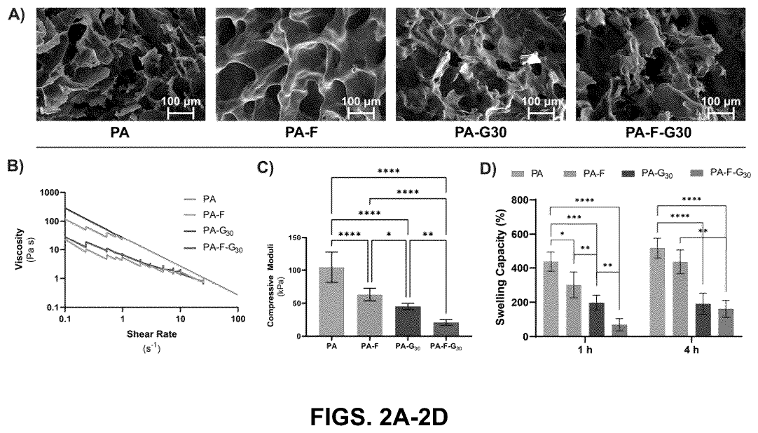

The cross-sectional morphology of the hydrogels was examined using SEM. Porosity and macroscopic network structure are critical components to hydrogel composition as they allow for high swelling potentials and gas exchange between tissues and surrounding environments. Imaging showed the highly porous nature of the hydrogels (FIG. 2A) resulting from the temperature-dependent nanostructure and organization of the Pluronic-F127 micelles.³⁸ The ionic crosslinking of alginate with calcium chloride further affects porosity as the network of alginate chains is variably crosslinked throughout the polymeric structure, intertwined with Pluronic micelle domains. As all gel types underwent the same fabrication and crosslinking process, no significant network changes were seen across gel formulations.

### Rheology Testing

Rotational tests were conducted on the hydrogel formulations following ISO 3219 standards to determine the apparent steady-state (shear) viscosities under variable shear rate. As shown in FIG. 2B, the hydrogels exhibited shear-thinning behavior, with decreased viscosity under increased shear load. The incorporation of NaF into the hydrogel matrices resulted in decreased apparent viscosity with respect to non-loaded gels at both low- and mid-shear conditions. Furthermore, GSNO incorporation resulted in minor deviation in apparent viscosity in the low shear region (i.e. <1 s⁻¹) respective to fluoride incorporation. Previously, Abrami et al. demonstrated that similar PA gels composed of greater than 18 wt % Pluronic F127 exhibit dense aggregation and packing of Pluronic micelles into ordered domains within the crosslinked alginate network at 37° C.⁴¹ These thermo-responsive properties of Pluronic F127 enable loading of GSNO and NaF at chilled and room temperature conditions, with enhanced packing achieved at physiological conditions. However, since fluoride ions in the gel can interact both with GSNO and divalent ions (e.g. Ca²⁺), its incorporation can modulate the mechanical strength of the gels in a formulation dependent manner.

### Compression Testing

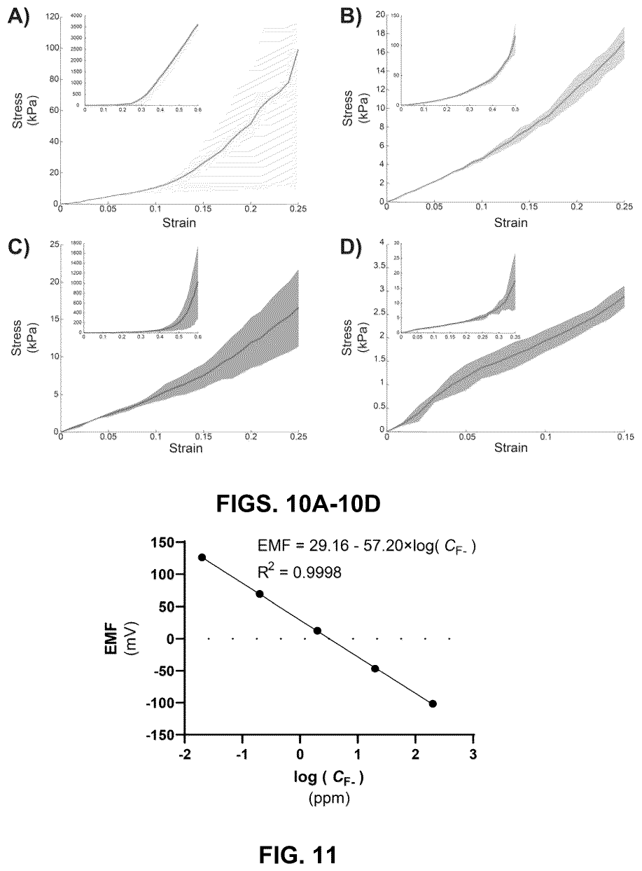

Compression moduli of hydrogels is an important property relating the stiffness of the material with its resistance to deformation under compressive load. In many soft tissue applications (e.g. gum tissue), compositional tunability of hydrogels is key to mimicking the complex viscoelastic properties of these tissues and for mediating controlled drug diffusion at the hydrogel-tissue interface. Uniaxial compression testing was performed on the developed PA gels at 25% strain at a rate of 0.166 mm s⁻¹ (FIG. 2C) to determine the mechanical properties in relation to gel composition based on stress-strain relationships (see FIG. 10). Under loading conditions, the PA control gels exhibited compression moduli of 104.4±23.3 kPa, with a statistically significant decrease (p<0.05) in moduli down to 20.99±4.175 kPa in the composite PA-F-G₃₀. These results parallel rheology findings, with NaF incorporation affecting the physical properties of the gels, possibly through interactions with divalent calcium ions used in the alginate crosslinking as well as interactions with GSNO that may affect micelle loading, packing, and stability. This high degree of compression moduli tunability based on GSNO and NaF incorporation parallels a need in soft tissue engineering applications for viscoelastic behavior tunable for tissue microenvironments.⁵¹ With these tunable mechanical properties, the further enhancement in NO and fluoride ion release was investigated.

### Swelling Capacity

Characterization of the water uptake capability can give further insight into the structure and physical properties of hydrogels. To simulate a physiological environment, all four hydrogel types were incubated in artificial saliva for 1 h and 4 h. The swelling capacity was calculated based on the mass of the gels after lyophilization. At both time points there was a noticeable trend between swelling capacity and hydrogel makeup. As components were added to the hydrogel (NaF and GSNO), the swelling capacity decreased, with PA-F-G₃₀ hydrogels swelling the least amount of artificial saliva at 68.86% at 1 h and 161.038% at 4 h (FIG. 2D). Control gels on the other hand swelled 436.7% and 517.15% at 1 h and 4 h, respectively. The hydrogels with only one component exhibited better swelling than PA-F-G₃₀, with PA-F retaining 301.77% and 436.2% at hours 1 and 4, while PA-G₃₀ swelled 197.59% at 1 h and 190.95% at 4 h. This relationship mirrors the results from compressive testing, where the addition of NaF, GSNO, and both can lead to decreased mechanical strength. This is likely attributable to divalent cation interactions with fluoride, which affect Ca²⁺ availability for ionic interactions with alginate responsible for crosslinking and structural stability of the gels. Although the treatment gel (PA-F-G₃₀) reveals the lowest mechanical strength and swelling capacity, soft tissue applications do not require extensive mechanical properties and these characteristics will not hinder the function of the designed hydrogel.

Chemiluminescence-Based Measurements of NO Release from Hydrogels

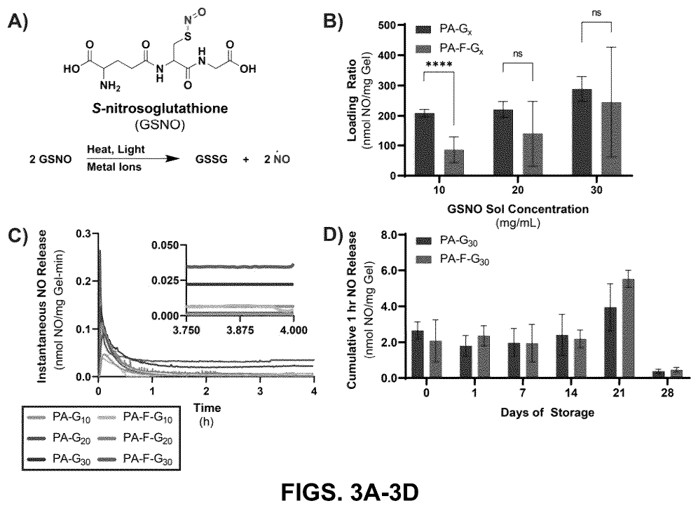

NO-releasing hydrogels have previously been developed for dermal wound healing,^{37, 52-53} promotion of angiogenesis,⁵⁴ and as stem cell carriers for treating myocardial infarction, hindlimb ischemia, and other illnesses.⁵⁵⁻⁵⁷ Prior work concerning Pluronic F127-alginate hydrogels with GSNO have demonstrated robust efficacy in a non-crosslinked form for dermal wound healing.⁵³ Similarly, previous work with NO-releasing hyperbranched polymers and silica nanoparticles have demonstrated the long-term efficacy of the material class towards resolving microbial adhesion and subsequent biofilm formation onto dental implants.⁵⁸⁻⁵⁹ GSNO is frequently used in hydrogels and other hydrophilic environments due to its favorable stability in aqueous conditions and ready liberation of NO in the presence of heat, light, or metal ions (FIG. 3A).⁶⁰ Herein we report for the first time the application of a NO-releasing hydrogel material to address both bacterial proliferation at dental soft tissue surfaces

### GSNO Loading

After fabrication, a vigorous washing step with PBS (1×) removes unbound GSNO from the gels, implicating that a significant amount of GSNO in the precursor sol may not be incorporated in the final gel. To assess the loading potential of GSNO into PA-Gₓ and PA-F-Gₓ gels, NO was liberated from GSNO incorporated within the crosslinked gels through the presence of Cu¹⁺ in a reductive environment. NO loading analysis using chemiluminescence-based NO detection showed that the initial GSNO concentration in the sol precursor significantly affected final loading in gels (FIG. 3B). While higher concentrations of GSNO in PA-G₂₀ and PA-G₃₀ led to no statistically significant difference in loading when NaF was incorporated, the difference for PA-G₁₀ was more pronounced with an almost 50% decrease in GSNO loading of PA-F-G₁₀ gels compared to PA-G₁₀ (p<0.0001). Considering the theoretical loading potential of GSNO into each gel based on the precursor sol concentration, this trend is mirrored with the PA-G₁₀ gel showing almost 85% loading efficiency, while all other GSNO-based gels had approximately 40-50% loading efficiency (Table 1). These results in combination with trends in the mechanical integrity of the gels (FIG. 2B) implicate GSNO and NaF-dependent structuring of the hydrogels, perhaps by influencing Pluronic micellization behavior. Previous work with GSNO-containing, non-crosslinked F127-alginate gels demonstrated that GSNO accelerates micellization in the process of temperature-dependent gelation.⁵³ Therefore, we believe the differences in GSNO loading capacities for PA-G₁₀ and PA-F-G₁₀ to be the result of different solubilization capacities when the presence of fluoride ions can both affect the dominant microspecies of GSNO in the gel as well as alginate crosslinking via interaction with calcium ions.⁶¹

TABLE 1
Sol Precursor Composition (By Mass Fraction, x)

| Formulation | x_{F127} | x_{Alginate} | x_{GSNO} | x_{NaF} | x_{Water} |
|---|---|---|---|---|---|
| PA | 0.150 | 0.0167 | 0.00000 | 0.00000 | 0.833 |
| PA-F | 0.150 | 0.0166 | 0.00000 | 0.00166 | 0.832 |
| PA-G₁₀ | 0.149 | 0.0165 | 0.00826 | 0.00000 | 0.826 |
| PA-F-G₁₀ | 0.149 | 0.0165 | 0.00825 | 0.00165 | 0.825 |
| PA-G₂₀ | 0.148 | 0.0164 | 0.0164 | 0.00000 | 0.820 |
| PA-F-G₂₀ | 0.147 | 0.0164 | 0.0164 | 0.00164 | 0.818 |
| PA-G₃₀ | 0.146 | 0.0163 | 0.0244 | 0.00000 | 0.813 |
| PA-F-G₃₀ | 0.146 | 0.0162 | 0.0244 | 0.00162 | 0.812 |

Differences in GSNO loading potential across different sol concentrations of GSNO were also investigated up to 30 mg/mL of GSNO, after which incomplete solubility of GSNO prohibited further loading evaluation at higher concentrations (FIG. 3B). Whereas no significant difference in GSNO loading was obtained between PA-G₁₀ and PA-G₂₀, increased loading was observed between PA-G₂₀ and PA-G₃₀ (p<0.01). Among fluoride-containing gels, differences were much more pronounced, with each incremental increase in GSNO sol concentration leading to at least a 75% increase in GSNO loading in the gel (p<0.0001). In each instance, increased loading can be attributed to the higher GSNO sol concentration, with fluoride ions potentially affecting GSNO incorporation during the micellization process and having further ionic interaction with Ca²⁺ found in the crosslinked alginate network.

### NO Release Under Physiological Conditions

Representative profiles of the NO release for each hydrogel formulation (compositions summarized in Table 1) were determined over an initial four hours after fabrication under physiological conditions (FIG. 3C). On average, PA-F-G₃₀ exhibited the highest release rates of NO over the study duration, with overall trends in NO release being strongly dependent on GSNO concentration in the sol precursor. The cumulative NO loadings of the representative spectra shown in FIG. 3C are summarized in Table 3 for each hour of the study. By the fourth hour of the study, in all cases the fluoride-containing formulation at a given GSNO concentration had achieved higher cumulative NO release than its respective GSNO only counterpart. These results are consistent with mechanical findings, demonstrating that fluoride incorporation affects the elasticity and crosslinking of the gel, which may lead to greater GSNO availability at the physiological interface and therefore increased NO release. Initially enhanced NO release from the fluoride-containing gels is beneficial from a therapeutic perspective, especially for combatting opportunist pathogens at the center of periodontal disease.

TABLE 2
Gel Loading Efficiency

| | Theoretical | Experimental | Loading |
|---|---|---|---|
| | GSNO Loading | NO Loading | Efficiency |
| Formulation | (nmol GSNO/mg Gel) | (nmol NO/mg Gel) | (%) |
| PA-G₁₀ | 246 | 209 ± 12.2 | 84.9 ± 4.94 |
| PA-F-G₁₀ | 245 | 103 ± 6.25 | 42.4 ± 2.55 |
| PA-G₂₀ | 487 | 220 ± 26.3 | 45.3 ± 5.41 |
| PA-F-G₂₀ | 487 | 198 ± 31.9 | 40.6 ± 6.55 |
| PA-G₃₀ | 725 | 288 ± 40.9 | 39.8 ± 5.64 |

TABLE 3
Cumulative 4 h NO Release (mean, nmol NO/mg gel)

| Formulation | Hour 1 | Hour 2 | Hour 3 | Hour 4 |
|---|---|---|---|---|
| PA-G₁₀ | 1.02 | 1.17 | 1.25 | 1.32 |
| PA-F-G₁₀ | 0.749 | 0.919 | 1.08 | 1.40 |
| PA-G₂₀ | 1.71 | 1.87 | 1.96 | 2.06 |
| PA-F-G₂₀ | 2.14 | 2.61 | 2.98 | 3.32 |
| PA-G₃₀ | 3.01 | 4.28 | 5.48 | 6.79 |
| PA-F-G₃₀ | 2.97 | 4.91 | 6.79 | 8.83 |

### Storage Stability Analysis (28 d)

The cumulative 1 h NO release from PA, PA-F, PA-G₃₀, and PA-F-G₃₀ after storage at 4° C. was investigated to determine the storage stability of the gels in terms of maintaining the NO release and corresponding antimicrobial efficacy. The gels were tested after storage conditions of 0, 1, 7, 14, 21, and 28 d. All gels were made on day 0 and N=5 for each sample type were removed, analyzed, and discarded on each measurement day. Cumulative 1 h NO release from PA-G₃₀ was 2.65 nmol/mg on day 0 followed by 1.80, 1.97, and 2.41 nmol/mg on days 1, 7, and 14, respectively. Similarly, PA-F-G₃₀ released 2.08, 2.37, 1.95, and 2.19 nmol/mg on days 0, 1, 7, and 14 (FIG. 3D). There was no significant difference between the cumulative 1 h NO release of PA-G₃₀ and PA-F-G₃₀ for the first 14 d, indicating the gels were able to maintain initial NO release and antimicrobial potential for at least 14 d when stored at 4° C. and in dark conditions. However, day 21 showed an increase in NO release from both sample types as PA-G₃₀ released 3.95 nmol/mg and PA-F-G₃₀ released 5.52 nmol/mg. The boost in NO release on day 21 is believed to be due to alginate degradation within the gels, leading to the less controlled GSNO decomposition and NO release, as GSNO is no longer bound by the polymeric matrix formed by the crosslinked Pluronic-alginate structure. The NO release on day 28 of storage at 4° C. confirms the degradation hypothesis, as much of the hydrogel structure has been lost by that time point and very little GSNO is remaining in the polymeric matrix, leading to a release of only 0.376 nmol/mg from PA-G₃₀ and 0.454 nmol/mg from PA-F-G₃₀.

### Fluoride Release

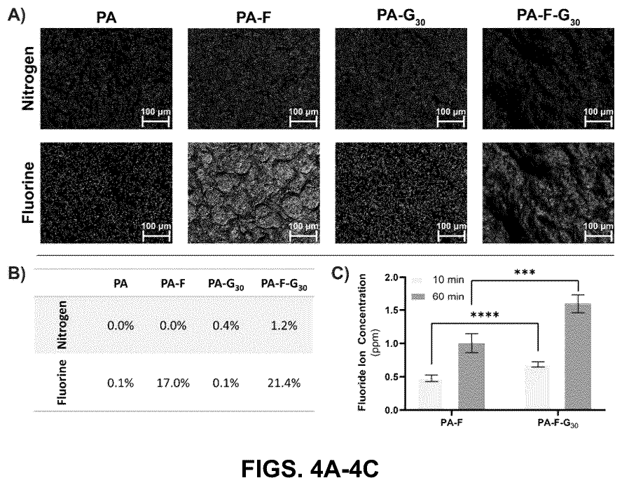

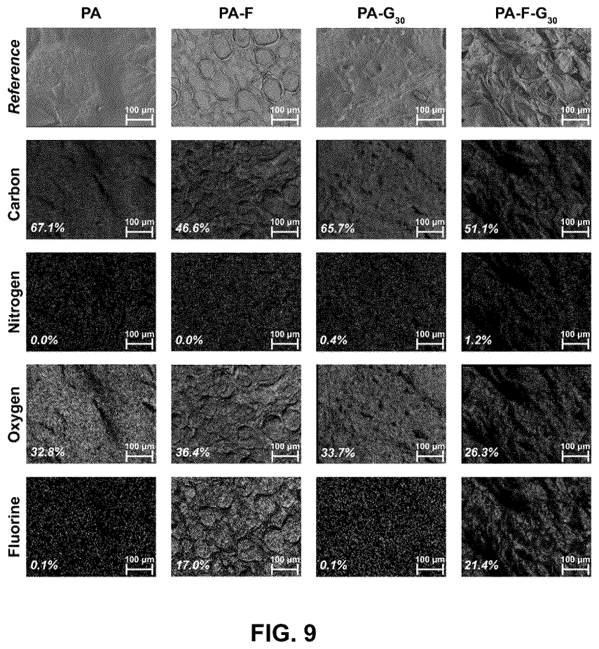

Fluoride ion release was assessed from representative PA-F and PA-F-G₃₀ gels to evaluate the capacity for leaching under physiological conditions to mediate processes of enamel demineralization prevention. EDS-SEM analysis of the various gel formulations demonstrated surface distribution of fluorine across both PA-F and PA-F-G₃₀ gels, confirming its integration into the hydrogel matrix (FIG. 4A). Full EDS spectra are provided in FIG. 9. Differences in fluorine surface distribution (FIG. 4B) between PA-F (17%) and PA-F-G₃₀ (21.4%) are likely attributable to ionic interactions between GSNO and fluoride ions in PA-F-G₃₀ gels, allowing for a greater number of fluoride ions to become captured into the polymeric structure. Furthermore, the surface crosslinking strategy utilized in this study may have led to a higher distribution of fluorine on the surface of the gels than what is maintained throughout the hydrogel network as calcium ions (present in the CaCl₂) crosslinking solution) are capable of deactivating fluorine ions through precipitation at the surface where crosslinking occurs.¹⁸ However, the surface-localized fluorine induced by the chosen crosslinking method may enhance the demineralization prevention effects, as the fluoride is made more available to the exposed hydroxyapatite/enamel, leading to augmented fluorapatite formation and greater demineralization prevention.

In addition to surface characterizations, fluoride ions released from the gel were further quantified. Using a fluoride ion-selective electrode, leachates from gels were evaluated after ten- and sixty-minutes incubation under physiological conditions (FIG. 4C), with fluoride ion concentration calculated against a standard curve from sodium fluoride (FIG. 11). On a basis of 0.2% w/w fluoride loading, an average of 0.476 and 1.002 PPM fluoride ions was detected in solution after ten minutes of leaching, with each increasing by over 40% after one hour. In both instances, PA-F-G₃₀ exhibited greater leaching of fluoride than PA-F alone (p<0.001). We justify these observations given the possibility for further ionic interactions with GSNO in PA-F-G₃₀ than PA-F alone, as well as the increased surface localization of fluorine in PA-F-G₃₀ (FIG. 4B). GSNO has one primary amine and two secondary amide groups that are readily protonated under physiological conditions, especially in the slightly acidic environment of artificial saliva (pH ˜ 6.8), for the formation of amine fluorides (FIG. 1C).⁶² A similar mechanism of controlled fluoride release is accomplished with other amine fluorides such as Olaflur, which utilize a surfactant component to adsorb as monolayers onto enamel and elicit a controlled release of fluoride at the tissue interface in addition to inducing a potent bactericidal effect with its residual cationic charge. 18 By mirroring these characteristics with GSNO loaded micelles in the PA-F-Gₓ gels, the material class showed remarkable biological properties after further evaluation.

Antimicrobial Evaluation. Planktonic Bacterial Viability Study

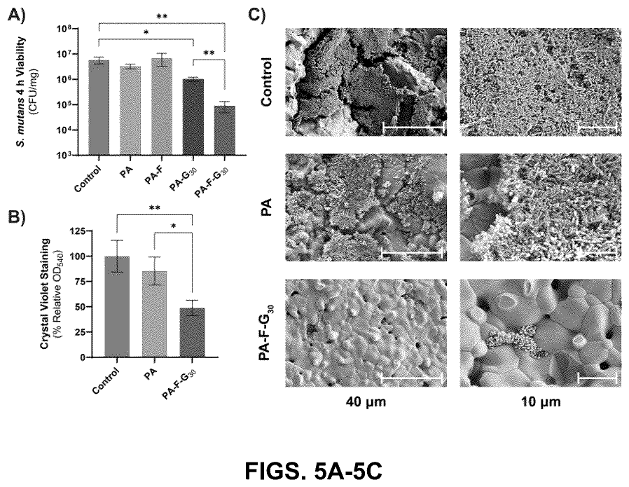

The antimicrobial potential of the NO releasing hydrogels was tested against S. mutans, a dental bacterium commonly found in the grooves and fissures of teeth. The excess colonization of S. mutans in the oral cavity can lead to an overproduction of acidic metabolic byproducts that are responsible for enamel decay and cavities. Therefore, a dental treatment that can effectively kill the microorganisms can help control the overproduction of harsh acids and deter tooth demineralization. At the same time, the fluoride released from PA-F-G₃₀ can help rebuild HA structures into more resilient enamel constructs that are less likely to decay if the bacterial infection were to return. As the application of the hydrogel in translational settings would be short term, 4 h incubation of the hydrogels in an S. mutans solution was investigated (FIG. 5A). As expected, PA and PA-F gels were not able to effectively kill bacteria, while gels incorporated with GSNO demonstrated a significant antimicrobial outcome. The antimicrobial effects of NO released from GSNO are due to the highly reactive nature of the free radical molecule and the production of other reactive oxygen (ROS) and nitrogen species (RNS) such as peroxynitrite (OONO⁻), nitrogen dioxide (NO₂^{•}), and hydrogen peroxide (H₂O₂).²² These molecules are able to penetrate the bacterial membrane and permanently damage lipids, transport proteins, DNA and DNA repair systems, as well as inactivate heme proteins responsible for signal transduction, ultimately leading to bacterial death.²² The bacteria treated with PA and PA-F showed no significant decrease in viable colony counts compared to control bacteria, which was expected because alginate, Pluronic F127, and fluoride contain no active antimicrobial mechanism of action. On the other hand, the NO release from PA-G₃₀ resulted in an 82.22% bacterial reduction, and PA-F-G₃₀ showed a 98.43% reduction of viable bacteria compared to controls. The increase in killing is attributed to the greater GSNO loading ratio and subsequent increase in released NO from PA-F-G₃₀. Greater NO release leads to higher levels of ROS and RNS in the bacterial environment, initiating membrane rupture, bacterial inactivation through protein and lipid disruption, and consequent S. mutans death. Furthermore, recent studies have shown that NO is more effective at killing S. mutans in acidic conditions that would be present if an in vivo infectious oral cavity were being treated.³⁴

S. mutans Biofilm Dispersal

In addition to inducing potent antimicrobial effects through the production of highly reactive ROS and RNS, NO is also capable of dispersing biofilms through penetration of the extracellular polymeric substance (EPS) and disruption of quorum sensing, or bacterial communication and adaptation within a biofilm.⁶³

The ability of NO to penetrate biofilms is uniquely attributable to its gaseous nature, a feature that antibiotics do not possess and therefore makes them significantly less effective at infiltrating, dispersing, and killing bacteria within a biofilm. Crystal violet staining is a technique widely used in biomedical research to quantify biofilms as the dye binds to negatively charged molecules present in bacteria and their surrounding EPS matrix.⁶⁴ Herein, crystal violet staining was used to quantify S. mutans biofilms grown on HA discs. The use of HA as a model for in vitro tooth enamel studies is well recognized and accepted since HA is the mineral that makes up 95-98% of teeth.⁷ Following the growth of a S. mutans biofilm on HA discs for 36 h, the treatment with PA and PA-F-G₃₀ gels for 24 h demonstrated the ability of released NO to decrease biofilm structure by 52% compared to control, untreated biofilms (FIG. 5B). PA gels reduced the biofilms slightly, but not by a significant amount. SEM imaging of the biofilms shown in FIG. 5C revealed the dense, interconnected S. mutans biofilms formed on the HA surface of control discs and those treated with PA gels. HA covered biofilms treated with PA-F-G₃₀ displayed a greater reduction in biomass, with S. mutans only surviving in deeper crevices of HA discs. However, compared to PA treated discs, those treated with PA-F-G₃₀ had fewer pores, showing the necessity for the fluoride component in the gels to decrease possible sites for bacterial invasion, a concept more heavily explored in the demineralization study. Overall, the CV biofilm quantification and SEM imaging of biofilms demonstrated the ability of NO to penetrate and disperse pre-formed S. mutans biofilms, validating the use of PA-F-G₃₀ gels to treat future cases of dental caries by breaking down mature biofilms and killing viable dental pathogens.

### Demineralization of Hydroxyapatite Enamel Model

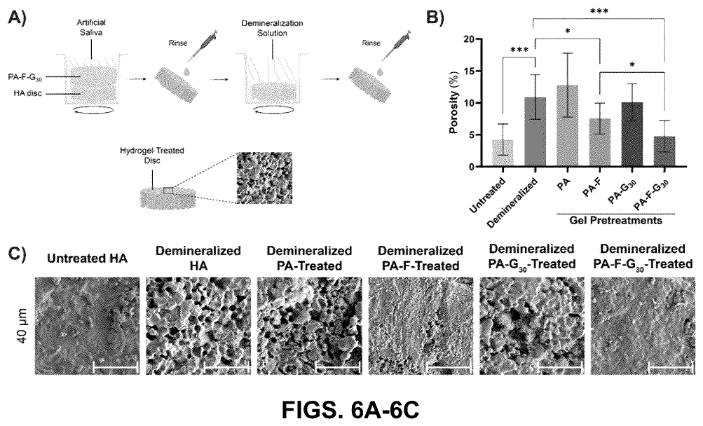

The treatment of tooth enamel with fluoride to prevent demineralization and strengthen damaged enamel structures has been used for almost a century through supplementation of city water supplies and recommended toothpaste and mouthwash products. The enamel restoration occurs when calcium and phosphate ions in saliva are disseminated into tooth enamel by fluoride ions. The influx of calcium and phosphate leads to recrystallization within cavities or demineralized portions of enamel and the formation of fluoridated HA, which is more impervious to acidic erosion than HA.¹ To mimic the physiological conditions of enamel demineralization, HA discs in artificial saliva solutions were treated with PA, PA-F, PA-G₃₀, and PA-F-G₃₀ followed by a highly acidic (pH=3) demineralization solution (FIG. 6A). The ability of the fluoride released from the gels to prevent HA demineralization was investigated by quantifying the induced porosity of HA discs (n=3 per sample type) after no gel treatment or treatment with each of the four gel types (FIGS. 6B and 6C). Porosity was correlated to demineralization, with higher porosity values corresponding to a greater extent of demineralization, which fluoride releasing gels sought to prevent. SEM images and single-blinded porosity quantification demonstrated the effective prevention of demineralization. Untreated HA discs (i.e., no gel or demineralization solution) had an average porosity of 4.25% while discs treated with PA-F-G₃₀ followed by demineralization solution had a porosity of 4.76%, indicating almost no change in porosity (demineralization) compared to the negative control even after treatment with a highly acidic demineralization solution. Treatment with PA-F prior to demineralization led to the next lowest porosity value at 7.54%, which correlated with fluoride ion release measurements, leading to greater demineralization protection with PA-F-G₃₀. Treatment with PA-F led to some protection, however, as HA not treated with hydrogel but incubated in demineralization solution resulted in 10.92% porosity, while HA treated with PA and acidic conditions showed 12.78% porosity. Incubation with PA-G₃₀ was also unsuccessful at preventing demineralization, with a porosity value of 10.10%. When comparing all treatment types to HA discs that did not undergo the demineralization process, the gels that prevented demineralization most successfully were PA-F-G₃₀ followed by PA-F, with surface structures most like native HA (FIG. 6C). On the other hand, PA and PA-G₃₀ provided essentially no protection as seen by the numerous gaps and cavities found on the surface of the HA structure. The difference in demineralization protection potential is therefore due to the release of fluoride ions that allow for restructuring and strengthening of HA microstructures through the capture of calcium and phosphate found in the artificial saliva. Without the fluoride release, HA is prone to fracture and demineralization in an acidic environment caused by food and overactive oral bacteria.

Cytocompatibility Evaluation. Cellular Cytotoxicity of GSNO and Precursor Sol Materials

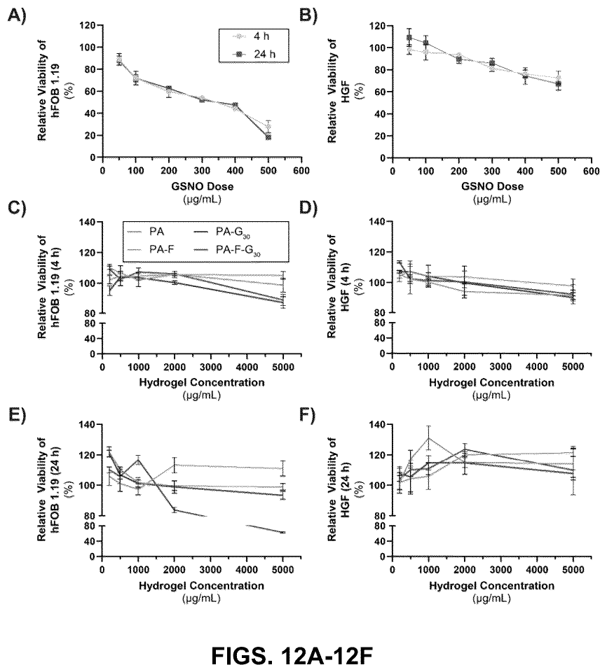

Drug-releasing hydrogels with degradable backbones such as alginate have attracted significant attention in recent years for tissue engineering and other therapeutic applications but can present issues if the drug release rate and degradation products induce a cytotoxic response. For this reason and to establish a baseline for further biological evaluation, the non-crosslinked hydrogel precursors as well as GSNO were evaluated for cytotoxic response in two representative human cell types: HGF and hFOB 1.19. In the oral cavity, fibroblasts are critical for developing the structural framework of connective tissue and propagating processes of inflammation and wound healing. Similarly, osteoblastic cells are essential to hard and soft tissue reconstruction following periodontal disease. During 4 and 24 h in vitro direct contact exposure studies, representative cytotoxic response curves were generated for both GSNO and the dissolved precursors (FIG. 12). GSNO elicited a controllable cytotoxic response at greater than 100 μg/mL treatments in HGFs, while the same was shown at nearly 400 μg/mL of GSNO in hFOB 1.19 cells. These results agree with literature, as HGFs are known to produce pM levels of NO for cellular signaling, while experiencing cytotoxic response at mM levels in response to periodontal disease.⁶⁵ Similarly, osteoblasts are known to respond to low levels of NO in the processes of bone remodeling⁶⁶ but can undergo apoptosis at higher levels.⁶⁷ For these reasons, NO donors are frequently embedded into polymeric materials to control their diffusion and degradation rates.⁶⁸

Further evaluation for cytotoxic response from the precursor hydrogels over 4 and 24 h in HGF and hFOB 1.19 showed minimal induction of a cytotoxic response (FIG. 125C-12F). In most cases, higher concentrations of the PA-G₃₀ and PA-F-G₃₀ hydrogel induced a mild cytotoxic response, in support of previous observations with increased GSNO concentrations. The presence of fluoride in PA-F and PA-F-G₃₀ even at higher doses did not significantly affect toxicity, supporting the use of NaF in the hydrogel composition without further concern for sodium fluoride-induced toxicity.⁶⁹

### Cell Proliferation in Presence of Hydrogel

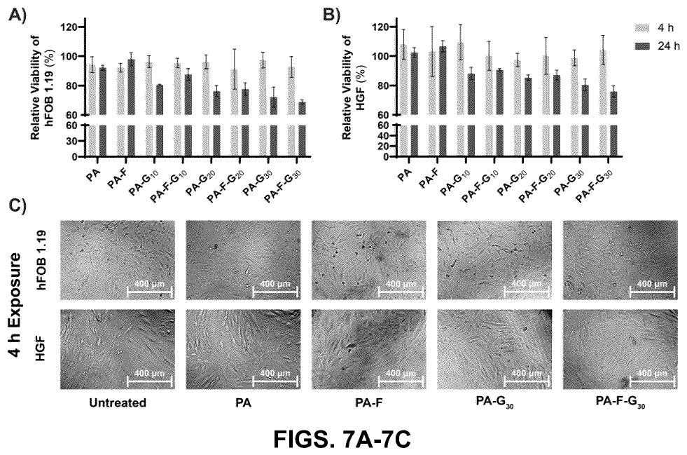

To further evaluate the cytocompatibility of the crosslinked AP gels, HGF and hFOB 1.19 were exposed to the various gel formulations over 4 and 24 h in direct contact studies. Alginate gels are quickly crosslinked using divalent cations such as Ca²⁺ through ionic interaction stabilization between neighboring strand carboxylic acid groups forming chain-chain associations. This added structural rigidity helps in controlling the diffusion of drugs through alginate networks. Although alginate is biologically inert, its gradual degradation may present issues for cytotoxicity, especially through increased calcium ion availability.⁷⁰ Mirroring further antibacterial studies, cells were exposed to 8 mm circular hydrogel film punches for 4 and 24 h. Throughout these studies, no considerable cytotoxic response was observed with any of the crosslinked gel formulations (FIGS. 7A and 7B). Increased GSNO loading was observed to elicit a mild decrease in cell proliferation, with these effects attenuated by the presence of fluoride ions in the films. Further examination of cells treated with gels for 4 h via brightfield microscopy showed no substantial differences in cell morphology, with few dead cells present proportional to the relative cytotoxicity (FIG. 7C). Taken all together, these results are justified by our prior mechanical testing and NO studies, which show that PA-F-G₃₀ are softer with greater NO release and loading compared to the other gels. By controlling the GSNO content of the gels, key mechanical and biological properties can be controlled for desired effect, as evaluated further in antimicrobial studies.

### CONCLUSION

In summary, we have investigated the feasibility and efficacy of a first-of-its kind NO- and fluoride ion-releasing hydrogel with highly tunable biological properties suitable for combatting pathogens at the root of periodontal disease. This novel class of dental hydrogels exhibited porous nanostructures with tunable mechanical properties based on GSNO and NaF incorporation. This design enables tailoring of the material to application-specific circumstances for dental soft tissue, with shear-thinning behavior suitable for rapid self-healing. Measurements of NO release studied in the first four hours revealed release in the nanomolar range, with gels retaining stability for over 14 d. Fluoride studies showed ppm fluoride ion release that was enhanced with the addition of GSNO to the gel. By adopting crosslinked alginate as a stabilizing network with temperature responsive F127 micelle structuring, GSNO and NaF are both incorporated into the hydrogel with controlled NO and fluoride ion release under physiological conditions.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

(1) Selwitz, R. H.; Ismail, A. I.; Pitts, N. B. Dental Caries. Lancet 2007, 369 (9555), 51-9.
(2) Oral-B What Are Dental Caries? Treatments, Signs, and Symptoms. https://oralb.com/en-us/oral-health/conditions/cavities-tooth-decay/what-are-dental-caries/.
(3) Us Dental Expenditures; Health Policy Institute: 2017.
(4) Jaffer, I. H.; Weitz, J. I. The Blood Compatibility Challenge. Part 1: Blood-Contacting Medical Devices: The Scope of the Problem. Acta Biomater 2019, 94, 2-10.
(5) Fluoridated Water. https://www.cancer.gov/about-cancer/causes-prevention/risk/myths/fluoridated-water-fact-sheet.
(6) Control, C. f. D. Community Water Fluoridation. https://www.cdc.gov/fluoridation/index.html.
(7) Enamel Structure and Composition.
(8) Murakami, C.; Bonecker, M.; Correa, M. S.; Mendes, F. M.; Rodrigues, C. R. Effect of Fluoride Varnish and Gel on Dental Erosion in Primary and Permanent Teeth. Arch Oral Biol 2009, 54 (11), 997-1001.
(9) Calvo, A. F.; Tabchoury, C. P.; Del Bel Cury, A. A.; Tenuta, L. M.; da Silva, W. J.; Cury, J. A. Effect of Acidulated Phosphate Fluoride Gel Application Time on Enamel Demineralization of Deciduous and Permanent Teeth. Caries Res 2012, 46 (1), 31-7.
(10) Wei, S. H.; Chik, F. F. Fluoride Retention Following Topical Fluoride Foam and Gel Application. Pediatr Dent 1990, 12 (6), 368-74.
(11) Kim, M.; Kwon, H.; Choi, C. H.; Kim, B. In Combined Effects of Nano-Hydroxyapatite and Naf on Remineralization of Early Caries Lesion, Key Engineering Materials, Trans Tech Publ: 2007; pp 1347-1350.
(12) Wiegand, A.; Krieger, C.; Attin, R.; Hellwig, E.; Attin, T. Fluoride Uptake and Resistance to Further Demineralisation of Demineralised Enamel after Application of Differently Concentrated Acidulated Sodium Fluoride Gels. Clinical oral investigations 2005, 9 (1), 52-57.
(13) Marinho, V. C.; Worthington, H. V.; Walsh, T.; Chong, L. Y. Fluoride Gels for Preventing Dental Caries in Children and Adolescents. Cochrane Database Syst Rev 2015, (6), CD002280.
(14) Weyant, R. J.; Tracy, S. L.; Anselmo, T. T.; Beltran-Aguilar, E. D.; Donly, K. J.; Frese, W. A.; Hujoel, P. P.; lafolla, T.; Kohn, W.; Kumar, J.; Levy, S. M.; Tinanoff, N.; Wright, J. T.; Zero, D.; Aravamudhan, K.; Frantsve-Hawley, J.; Meyer, D. M.; American Dental Association Council on Scientific Affairs Expert Panel on Topical Fluoride Caries Preventive, A. Topical Fluoride for Caries Prevention: Executive Summary of the Updated Clinical Recommendations and Supporting Systematic Review. J Am Dent Assoc 2013, 144 (11), 1279-91.
(15) Fejerskov, O.; Kidd, E. Dental Caries: The Disease and Its Clinical Management, John Wiley & Sons: 2009.
(16) Featherstone, J. D. Dental Caries: A Dynamic Disease Process. Aust Dent J 2008, 53 (3), 286-91.
(17) Forssten, S. D.; Bjorklund, M.; Ouwehand, A. C. Streptococcus Mutans, Caries and Simulation Models. Nutrients 2010, 2 (3), 290-8.
(18) Epple, M.; Enax, J. Moderne Zahnpflege Aus Chemischer Sicht. Chemie in unserer Zeit 2018, 52 (4), 218-228.
(19) Baehni, P. C.; Takeuchi, Y. Anti-Plaque Agents in the Prevention of Biofilm-Associated Oral Diseases. Oral Dis 2003, 9 Suppl 1, 23-9.
(20) Shapira, L.; Tepper, P.; Steinberg, D. The Interactions of Human Neutrophils with the Constituents of an Experimental Dental Biofilm. J Dent Res 2000, 79 (10), 1802-7.
(21) Anggard, E. Nitric Oxide: Mediator, Murderer, and Medicine. Lancet 1994, 343 (8907), 1199-206.
(22) Fang, F. C. Perspectives Series: Host/Pathogen Interactions. Mechanisms of Nitric Oxide-Related Antimicrobial Activity. J Clin Invest 1997, 99 (12), 2818-25.
(23) Furchgott, R. F.; Zawadzki, J. V. The Obligatory Role of Endothelial Cells in the Relaxation of Arterial Smooth Muscle by Acetylcholine. Nature 1980, 288 (5789), 373-6.
(24) Garren, M. R.; Ashcraft, M.; Qian, Y.; Douglass, M.; Brisbois, E. J.; Handa, H. Nitric Oxide and Viral Infection: Recent Developments in Antiviral Therapies and Platforms. Applied Materials Today 2021, 22, 100887.
(25) Wink, D. A.; Kasprzak, K. S.; Maragos, C. M.; Elespuru, R. K.; Misra, M.; Dunams, T. M.; Cebula, T. A.; Koch, W. H.; Andrews, A. W.; Allen, J. S.; et al. DNA Deaminating Ability and Genotoxicity of Nitric Oxide and Its Progenitors. Science 1991, 254 (5034), 1001-3.
(26) De Groote, M. A.; Fang, F. C. No Inhibitions: Antimicrobial Properties of Nitric Oxide. Clinical Infectious Diseases 1995, 21 (Supplement_2), S162-S165.
(27) Hoiby, N.; Bjarnsholt, T.; Givskov, M.; Molin, S.; Ciofu, O. Antibiotic Resistance of Bacterial Biofilms. Int J Antimicrob Agents 2010, 35 (4), 322-32.
(28) Moncada, S.; Higgs, A. The L-Arginine-Nitric Oxide Pathway. The New England journal of medicine 1993, 329 (27), 2002-12.
(29) Carossa, S.; Pera, P.; Doglio, P.; Lombardo, S.; Colagrande, P.; Brussino, L.; Rolla, G.; Bucca, C. Oral Nitric Oxide During Plaque Deposition. Eur J Clin Invest 2001, 31 (10), 876-9.
(30) Pan, Z.; Guzeldemir, E.; Toygar, H. U.; Bal, N.; Bulut, S. Nitric Oxide Synthase in Gingival Tissues of Patients with Chronic Periodontitis and with and without Diabetes. J Periodontol 2010, 81 (1), 109-20.
(31) Carpenter, A. W.; Reighard, K. P.; Saavedra, J. E.; Schoenfisch, M. H. O 2-Protected Diazeniumdiolate-Modified Silica Nanoparticles for Extended Nitric Oxide Release from Dental Composites. Biomaterials science 2013, 1 (5), 456-459.
(32) Backlund, C. J.; Sergesketter, A. R.; Offenbacher, S.; Schoenfisch, M. H. Antibacterial Efficacy of Exogenous Nitric Oxide on Periodontal Pathogens. J Dent Res 2014, 93 (11), 1089-94.
(33) Backlund, C. J.; Worley, B. V.; Schoenfisch, M. H. Anti-Biofilm Action of Nitric Oxide-Releasing Alkyl-Modified Poly (Amidoamine) Dendrimers against Streptococcus Mutans. Acta Biomater 2016, 29, 198-205.
(34) Backlund, C. J.; Worley, B. V.; Sergesketter, A. R.; Schoenfisch, M. H. Kinetic-Dependent Killing of Oral Pathogens with Nitric Oxide. J Dent Res 2015, 94 (8), 1092-8.
(35) Pelegrino, M. T.; De Araujo Lima, B.; Do Nascimento, M. H. M.; Lombello, C. B.; Brocchi, M.; Seabra, A. B. Biocompatible and Antibacterial Nitric Oxide-Releasing Pluronic F-127/Chitosan Hydrogel for Topical Applications. Polymers (Basel) 2018, 10 (4).
(36) Champeau, M.; Povoa, V.; Militao, L.; Cabrini, F. M.; Picheth, G. F.; Meneau, F.; Jara, C. P.; de Araujo, E. P.; de Oliveira, M. G. Supramolecular Poly (Acrylic Acid)/F127 Hydrogel with Hydration-Controlled Nitric Oxide Release for Enhancing Wound Healing. Acta Biomater 2018, 74, 312-325.
(37) Vercelino, R.; Cunha, T. M.; Ferreira, E. S.; Cunha, F. Q.; Ferreira, S. H.; de Oliveira, M. G. Skin Vasodilation and Analgesic Effect of a Topical Nitric Oxide-Releasing Hydrogel. J Mater Sci Mater Med 2013, 24 (9), 2157-69.
(38) Shishido, S. M.; Seabra, A. B.; Loh, W.; Ganzarolli de Oliveira, M. Thermal and Photochemical Nitric Oxide Release from S-Nitrosothiols Incorporated in Pluronic F127 Gel: Potential Uses for Local and Controlled Nitric Oxide Release. Biomaterials 2003, 24 (20), 3543-53.
(39) Lee, K. Y.; Mooney, D. J. Alginate: Properties and Biomedical Applications. Prog Polym Sci 2012, 37 (1), 106-126.
(40) Grassi, G.; Crevatin, A.; Farra, R.; Guarnieri, G.; Pascotto, A.; Rehimers, B.; Lapasin, R.; Grassi, M. Rheological Properties of Aqueous Pluronic-Alginate Systems Containing Liposomes. J Colloid Interface Sci 2006, 301 (1), 282-90.
(41) Abrami, M.; D'Agostino, I.; Milcovich, G.; Fiorentino, S.; Farra, R.; Asaro, F.; Lapasin, R.; Grassi, G.; Grassi, M. Physical Characterization of Alginate-Pluronic F127 Gel for Endoluminal Nabds Delivery. Soft Matter 2014, 10 (5), 729-37.
(42) Mondal, A.; Douglass, M.; Hopkins, S. P.; Singha, P.; Tran, M.; Handa, H.; Brisbois, E. J. Multifunctional S-Nitroso-N-Acetylpenicillamine-Incorporated Medical-Grade Polymer with Selenium Interface for Biomedical Applications. ACS Appl Mater Interfaces 2019, 11 (38), 34652-34662.
(43) Fan, Y.; Wen, Z. T.; Liao, S.; Lallier, T.; Hagan, J. L.; Twomley, J. T.; Zhang, J. F.; Sun, Z.; Xu, X. Novel Amelogenin-Releasing Hydrogel for Remineralization of Enamel Artificial Caries. J Bioact Compat Polym 2012, 27 (6), 585-603.
(44) Oshiro, M.; Yamaguchi, K.; Takamizawa, T.; Inage, H.; Watanabe, T.; Irokawa, A.; Ando, S.; Miyazaki, M. Effect of Cpp-Acp Paste on Tooth Mineralization: An Fe-Sem Study. J Oral Sci 2007, 49 (2), 115-20.
(45) Valentijn-Benz, M.; van't Hof, W.; Bikker, F. J.; Nazmi, K.; Brand, H. S.; Sotres, J.; Lindh, L.; Arnebrant, T.; Veerman, E. C. Sphingoid Bases Inhibit Acid-Induced Demineralization of Hydroxyapatite. Caries Res 2015, 49 (1), 9-17.
(46) Grassi, G.; Farra, R.; Noro, E.; Voinovich, D.; Lapasin, R.; Dapas, B.; Alpar, O.; Zennaro, C.; Carraro, M.; Giansante, C. Characterization of Nucleic Acid Molecule/Liposome Complexes and Rheological Effects on Pluronic/Alginate Matrices. Journal of drug delivery science and technology 2007, 17 (5), 325-331.
(47) Hopkins, S. P.; Pant, J.; Goudie, M. J.; Nguyen, D. T.; Handa, H. Electrospun Bioabsorbable Fibers Containing S-Nitrosoglutathione for Tissue Engineering Applications. ACS Applied Bio Materials 2020, 3 (11), 7677-7686.
(48) Pant, J.; Pedaparthi, S.; Hopkins, S. P.; Goudie, M. J.; Douglass, M. E.; Handa, H. Antibacterial and Cellular Response toward a Gasotransmitter-Based Hybrid Wound Dressing. ACS Biomater Sci Eng 2019, 5 (8), 4002-4012.
(49) Diniz, I. M.; Chen, C.; Xu, X.; Ansari, S.; Zadeh, H. H.; Marques, M. M.; Shi, S.; Moshaverinia, A. Pluronic F-127 Hydrogel as a Promising Scaffold for Encapsulation of Dental-Derived Mesenchymal Stem Cells. J Mater Sci Mater Med 2015, 26 (3), 153.
(50) Saarai, A.; Kasparkova, V.; Sedlacek, T.; Saha, P. On the Development and Characterisation of Crosslinked Sodium Alginate/Gelatine Hydrogels. J Mech Behav Biomed Mater 2013, 18, 152-66.
(51) Xia, T.; Liu, W.; Yang, L. A Review of Gradient Stiffness Hydrogels Used in Tissue Engineering and Regenerative Medicine. J Biomed Mater Res A 2017, 105 (6), 1799-1812.
(52) Masters, K. S.; Leibovich, S. J.; Belem, P.; West, J. L.; Poole-Warren, L. A. Effects of Nitric Oxide Releasing Poly (Vinyl Alcohol) Hydrogel Dressings on Dermal Wound Healing in Diabetic Mice. Wound Repair Regen 2002, 10 (5), 286-94.
(53) Cao, J.; Su, M.; Hasan, N.; Lee, J.; Kwak, D.; Kim, D. Y.; Kim, K.; Lee, E. H.; Jung, J. H.; Yoo, J. W. Nitric Oxide-Releasing Thermoresponsive Pluronic F127/Alginate Hydrogel for Enhanced Antibacterial Activity and Accelerated Healing of Infected Wounds. Pharmaceutics 2020, 12 (10), 926.
(54) Zahid, A. A.; Ahmed, R.; Raza Ur Rehman, S.; Augustine, R.; Tariq, M.; Hasan, A. Nitric Oxide Releasing Chitosan-Poly (Vinyl Alcohol) Hydrogel Promotes Angiogenesis in Chick Embryo Model. Int J Biol Macromol 2019, 136, 901-910.
(55) Yao, X.; Liu, Y.; Gao, J.; Yang, L.; Mao, D.; Stefanitsch, C.; Li, Y.; Zhang, J.; Ou, L.; Kong, D.; Zhao, Q.; Li, Z. Nitric Oxide Releasing Hydrogel Enhances the Therapeutic Efficacy of Mesenchymal Stem Cells for Myocardial Infarction. Biomaterials 2015, 60, 130-40.
(56) Zhang, K.; Chen, X.; Li, H.; Feng, G.; Nie, Y.; Wei, Y.; Li, N.; Han, Z.; Han, Z. C.; Kong, D.; Guo, Z.; Zhao, Q.; Li, Z. A Nitric Oxide-Releasing Hydrogel for Enhancing the Therapeutic Effects of Mesenchymal Stem Cell Therapy for Hindlimb Ischemia. Acta Biomater 2020, 113, 289-304.
(57) Carpenter, A. W.; Schoenfisch, M. H. Nitric Oxide Release: Part Ii. Therapeutic Applications. Chem Soc Rev 2012, 41 (10), 3742-52.
(58) Carpenter, A. W.; Reighard, K. P.; Saavedra, J. E.; Schoenfisch, M. H. O (2)-Protected Diazeniumdiolate-Modified Silica Nanoparticles for Extended Nitric Oxide Release from Dental Composites. Biomater Sci 2013, 1 (5), 456-459.
(59) Reighard, K. P.; Schoenfisch, M. H. Antibacterial Action of Nitric Oxide-Releasing Chitosan Oligosaccharides against Pseudomonas Aeruginosa under Aerobic and Anaerobic Conditions. Antimicrob Agents Chemother 2015, 59 (10), 6506-13.
(60) Amadeu, T. P.; Seabra, A. B.; de Oliveira, M. G.; Costa, A. M. S-Nitrosoglutathione-Containing Hydrogel Accelerates Rat Cutaneous Wound Repair. J Eur Acad Dermatol Venereol 2007, 21 (5), 629-37.
(61) Shin, D. Y.; Cheon, K. H.; Song, E. H.; Seong, Y. J.; Park, J. U.; Kim, H. E.; Jeong, S. H. Fluorine-Ion-Releasing Injectable Alginate Nanocomposite Hydrogel for Enhanced Bioactivity and Antibacterial Property. Int J Biol Macromol 2019, 123, 866-877.
(62) Park, J.-W.; Means, G. E. An Unusually Stable S-Nitrosothiol from Glutathione. Archives of Pharmacal Research 1989, 12 (4), 257-258.
(63) Barraud, N.; Storey, M. V.; Moore, Z. P.; Webb, J. S.; Rice, S. A.; Kjelleberg, S. Nitric Oxide-Mediated Dispersal in Single- and Multi-Species Biofilms of Clinically and Industrially Relevant Microorganisms. Microb Biotechnol 2009, 2 (3), 370-8.
(64) Ommen, P.; Zobek, N.; Meyer, R. L. Quantification of Biofilm Biomass by Staining: Non-Toxic Safranin Can Replace the Popular Crystal Violet. J Microbiol Methods 2017, 141, 87-89.
(65) Daghigh, F.; Borghaei, R. C.; Thornton, R. D.; Bee, J. H. Human Gingival Fibroblasts Produce Nitric Oxide in Response to Proinflammatory Cytokines. J Periodontol 2002, 73 (4), 392-400.
(66) Chae, H. J.; Park, R. K.; Chung, H. T.; Kang, J. S.; Kim, M. S.; Choi, D. Y.; Bang, B. G.; Kim, H. R. Nitric Oxide Is a Regulator of Bone Remodelling. J Pharm Pharmacol 1997, 49 (9), 897-902.
(67) Kuzushima, M.; Mogi, M.; Togari, A. Cytokine-Induced Nitric-Oxide-Dependent Apoptosis in Mouse Osteoblastic Cells: Involvement of P38map Kinase. Arch Oral Biol 2006, 51 (11), 1048-53.
(68) Rong, F.; Tang, Y.; Wang, T.; Feng, T.; Song, J.; Li, P.; Huang, W. Nitric Oxide-Releasing Polymeric Materials for Antimicrobial Applications: A Review. Antioxidants (Basel) 2019, 8 (11), 556.
(69) Agalakova, N. I.; Gusev, G. P. Molecular Mechanisms of Cytotoxicity and Apoptosis Induced by Inorganic Fluoride. ISRN Cell Biology 2012, 2012, 1-16.
(70) Cao, N.; Chen, X. B.; Schreyer, D. J. Influence of Calcium lons on Cell Survival and Proliferation in the Context of an Alginate Hydrogel. ISRN Chemical Engineering 2012, 2012.

1. A composition comprising a poloxamer, an alginate, a nitric oxide releasing compound, fluoride ions, and calcium ions.

2. The composition of claim 1, wherein the poloxamer has the formula
HO(C₂H₄O)_{b}(C₃H₆O)ₐ(C₂H₄O)_{b}OH
wherein a is from 10 to 100 and b is from 5 to 250, 10 to 225, 20 to 200, 50 to 200, 100 to 200, or 150 to 200.

3. The composition of claim 1, wherein the poloxamer has a molecular weight of from about 1 kDa to about 20 kDa.

4. The composition of claim 1, wherein the poloxamer has a molecular weight from about 12,000 Da to about 13,000 Da, about 175 to about 225 ethylene oxide units, and about 50 to about 75 propylene oxide units.

5. The composition of claim 1, wherein the poloxamer is from about 10% weight/volume (w/v) to about 30% weight/volume (w/v) of the composition.

6. The composition of claim 1, wherein the alginate comprises an alkali metal alginate or an alkaline earth metal alginate.

7. The composition of claim 1, wherein the alginate comprises sodium alginate.

8. The composition of claim 1, wherein the alginate has a viscosity of from about 5 centipoise to about 50 centipoise.

9. The composition of claim 1, wherein the alginate is from about 0.1% weight/volume (w/v) to about 5% weight/volume (w/v) of the composition.

10. The composition of claim 1, wherein the weight/volume (w/v) ratio of poloxamer to alginate is from 1:1 to 20:1.

11. The composition of claim 1, wherein the nitric oxide releasing compound is a S-nitrosothiol compound.

12. The composition of claim 1, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

13. (canceled)

14. The composition of claim 1, wherein the nitric oxide releasing compound is from about 0.1 mg/ml of the composition to about 30 mg/ml of the composition.

15. The composition of claim 1, wherein the fluoride ions are from about 0.01% weight/volume (w/v) to about 1% weight/volume (w/v) of the composition.

16. (canceled)

17. A composition produced by the process comprising
(a) mixing an alginate, a nitric oxide releasing compound, and a fluoride salt in water to produce a first composition;
(b) mixing a poloxamer with the first composition to produce a second composition comprising a gel; and
(c) applying a solution comprising a calcium salt to the gel to produce the composition.

18. (canceled)

19. (canceled)

20. (canceled)

21. (canceled)

22. (canceled)

23. (canceled)

24. The composition of claim 1, wherein the composition comprises a hydrogel.

25. A method for delivering fluoride ions and nitric oxide to the oral cavity of a subject, the method comprising delivering the composition 1-24 of claim 1 to the oral cavity of the subject.

26. (canceled)

27. (canceled)

28. A method for treating or preventing a bacterial infection in an oral cavity of a subject in need thereof comprising administering to the subject the composition 1-24 of claim 1.

29. A method for treating or preventing a periodontal disease in a subject in need thereof comprising administering to the subject the composition 1-24 of claim 1.

30. A method for preventing or reducing the formation of biofilm or dental plaque in a subject in need thereof comprising administering to the subject the composition 1-24 of claim 1.

31-35. (canceled)
